(19) United States
(12) Patent Application Publication
(10) Pub. No.: US 2006/0057559 A1
(43) Pub. Date: Mar. 16, 2006

(54) High-throughput cell migration screening assay

(75) Inventors: Xiang Xu, South San Francisco, CA (US); Yuanming Hu, Redwood City, CA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Rigel Pharmaceuticals, Inc., San Francisco, CA (US)

(21) Appl. No.: 11/166,920

(22) Filed: Jun. 23, 2005

Related U.S. Application Data
(60) Provisional application No. 60/582,135, filed on Jun. 23, 2004.

(51) Int. Cl. C12Q 1/00; C12Q 1/68; G01N 33/53

(52) U.S. Cl. 435/4; 435/6; 435/7.1

(57) ABSTRACT

The invention relates to methods for identifying and using agents, including small organic molecules, antibodies, peptides, cyclic peptides, nucleic acids, antisense nucleic acids, sphingolipid analogs, and ribozymes, that modulate cell activation or migration, e.g., lymphocyte migration, via modulation of the expression and/or activity of migration molecules such as, for example, EDG molecules (e.g., EDG1 and EDG3), selectins, integrins, cadherins, certain members of the immunoglobulin superfamily of molecules, or chemokine receptor molecule. The methods of the invention are efficient and readily amenable to high-throughput drug screening protocols. High-throughput screening (HTS) methods, compositions, and kits for performing the assays are also provided.

### RELATED APPLICATIONS

This application claims the benefit of priority to U.S. Provisional Patent Application Ser. No. 60/582,135, filed Jun. 23, 2004, all of which application is hereby incorporated by reference in its entirety.

### BACKGROUND OF THE INVENTION

Cell migration plays a central role in a wide variety of biological phenomena including embryonic development, angiogenesis, wound healing, immune response, and inflammation. In embryogenesis, cellular migrations are a recurring theme in important morphogenic processes ranging from gastrulation to development of the nervous system. In the adult organism, cell migration remains prominent in both physiological and pathological conditions. Migration of fibroblasts and vascular endothelial cells is essential for wound healing. In metastasis, tumor cells migrate from the initial tumor mass throughout the whole body. Directed tumor cell motility by chemotaxis is the final step of tumor invasion, and the modulation, e.g., inhibition of this process has been a major focus of research. Furthermore, it has been shown that αvβ3 and other cell adhesion molecules are involved in angiogenesis, bone turnover, and tumor cell proliferation (Nemeth J A et al. (2003) Clin Exp Metastasis. 20(5):413-20).

Cell migration and activation are also central in immune response. Lymphocytes play a number of crucial roles in immune responses, including direct killing of virus-infected cells, cytokine and antibody production, and facilitation of B cell responses. Lymphocytes are also involved in acute and chronic inflammatory disease; asthma; allergies; autoimmune diseases such as scleroderma, pernicious anemia, multiple sclerosis, myasthenia gravis, IDDM, rheumatoid arthritis, systemic lupus erythematosus, and Crohn's disease; and organ and tissue transplant disease, e.g., graft vs. host disease.

Identification of modulators of molecules which participate in cell migration and/or activation, including lymphocyte migration and activation, is important for developing therapeutic reagents which treat or prevent diseases or disorders associated with cell migration and/or activation. Accordingly, there is a need for efficient, high-throughput screening assays for use in identifying such modulators.

### SUMMARY OF THE INVENTION

The present invention is based, at least in part, on the development of assays for the identification of compounds which modulate cell migration, e.g., lymphocyte or endothelial cell migration or activation. The assays of the inventions may be carried out in a high throughput format and are readily amenable to automation.

Accordingly, in one aspect, the invention comprises a method for identifying a compound which modulates cell migration comprising contacting a cell which overexpresses a migration molecule with a test agent and a migration molecule ligand and measuring migration of the cell towards the ligand, wherein cell migration is modulated in the presence of the test agent as compared to in the absence of the test agent. In one embodiment, the cell stably overexpresses a migration molecule. In another embodiment, the cell transiently overexpresses a migration molecule. The cell may be, for example, an immune cell, e.g., a lymphocyte, or an endothelial cell. In one embodiment, the cell is a Jurkat cell.

In one embodiment, the migration molecule is an EDG molecule, e.g., EDG1 or EDG3. In another embodiment, the migration molecule is an immunoglobulin superfamily molecule. In another embodiment, the migration molecule is selected from the group consisting of: a chemokine receptor, a selectin, an integrin molecule, and a cadherin molecule. For example, a chemokine receptor molecule may be selected from the group consisting of: CCR1, CCR2, CCR3, CCR4, CCR5, CCR6, CCR7, CCR8, CCR9, CCR10, CCR11, CXCR1, CXCR2, CXCR3, CXCR4, CXCR5, CX3CR1, and XCR1. Migration molecules, as described herein, also include adhesion molecules, e.g., selectins and integrins. A selectin molecule may be selected from the group consisting of: L-selectin, E-selectin, and P-selectin. An integrin molecule may be selected from the group consisting of: α1β1, α2β1, α3β1, α4β1, α5β1, α6β1, α7β1, α8β1 (VLA-8), α9β1, αvβ3, αVβ1, αLβ2, αMβ2, αXβ2, αIIβ3, α6β3, α6β4, αVβ5, αVβ6, αVβ8, α4β7, αIELβ7, and α11. Exemplary immunoglobulin superfamily molecules include: Inter-Cellular Adhesion Molecule-1 (I-CAM-1) (CD54), Inter-Cellular Adhesion Molecule-2 (I-CAM-2) (CD 102), Inter-Cellular Adhesion Molecule-3 (I-CAM-3) (CD50), and Vascular-Cell Adhesion Molecule (V-CAM), ALCAM (CD166), Basigin (CD147), BL-CAM (CD22), CD44, Lymphocyte function antigen-2 (LFA-2) (CD2), LFA-3 (CD 58), Major histocompatibility complex (MHC) molecules, MAdCAM-1, PECAM (CD31). A cadherin molecule may be selected from the group consisting of: Cadherin E (1), Cadherin N (2), Cadherin BR (12), Cadherin P (3), Cadherin R (4), Cadherin M (15), Cadherin VE (5) (CD144), Cadherin T & H (13), Cadherin OB (11), Cadherin K (6), Cadherin 7, Cadherin 8, Cadherin KSP (16), Cadherin LI (17), Cadherin 18, Cadherin, Fibroblast 1 (19), Cadherin Fibroblast 2 (20), Cadherin Fibroblast 3 (21), Cadherin 23, Desmocollin 1, Desmocollin 2, Desmoglein 1, Desmoglein 2, Desmoglein 3, and Protocadherin 1, 2, 3, 7, 8, and 9. The molecules used in the methods of the invention are not limited to the molecules set forth above, and may include any migration molecule.

In a further embodiment, the ligand is sphingosine-1-phosphate (S1P). In one embodiment, a test agent used in the methods of the invention is selected from the group consisting of: a small organic molecule, polypeptide, antibody, nucleic acid, or lipid.

In one embodiment, the cells used in the methods of the invention may be lipid starved. In another embodiment, the cell contains a retroviral vector encoding said migration molecule. In a further embodiment, the cells are labeled, e.g., with a fluorescent dye, e.g., CyQuant GR™ dye. In another embodiment, cell migration is measured using a fluorescence plate reader. In still another embodiment, cell migration is measured at, for example, 485/530 nm.

In one embodiment, the cells are labeled after migration. In another embodiment, the cells are labeled prior to migration. In a further embodiment, the compound inhibits cell migration. In still another embodiment, the compound stimulates cell migration.

In another embodiment, the methods of the invention are carried out in a high-throughput format. For example, the method may be carried out in a vessel capable of holding multiple samples, e.g., in a 24-well, 48-well, 96-well, 384-well, or 1,536-well format to allow screening multiple test agents simultaneously. In another embodiment, the high throughput format is automated. In still another embodiment, each well contains a different test agent. In yet another embodiment, migration of the cell is from a first vessel to a second vessel, e.g., across a membrane.

In another aspect, the invention includes vectors which may be used to transform or transfect a cell in order to express a migration molecule. For example, the present invention includes a vector comprising a 5′ long terminal repeat (LTR), a reporter gene, the coding sequence of EDG1, a transcriptional response element (TRE), and a 3′ self-inactivating long terminal repeat (SIN-LTR). In one embodiment, an internal ribosome entry site (IRES) is inserted between the reporter gene and the coding sequence of EDG1. In another embodiment, the transcriptional response element (TRE) is a minimal promoter (Pmin). In another embodiment, the reporter gene is GFP.

Another example of a vector of the invention is a vector comprising an EF-1α promoter, a reporter gene, the coding sequence of EDG3, and a marker gene. In one embodiment, the marker gene is a resistance gene. In another embodiment, the resistance gene is neomycin. In still another embodiment, the reporter gene is GFP. In yet another embodiment, an internal ribosome entry site (IRES) is inserted between the reporter gene and the coding sequence of EDG 1.

The present invention also includes cells stably or transiently transformed or transfected with a vector containing a migration molecule of the invention, e.g., an immune cell, e.g., a lymphocyte, an endothelial cell, or a Jurkat cell.

### BRIEF DESCRIPTION OF THE DRAWINGS

FIG. 1 depicts examples of EDG1 and EDG3 constructs (“TIM EGD1 vector” and “pNIGEDG3 vector”) which may be used to generate EDG1 and EDG3 stable cell lines.

FIG. 2 is a graph depicting migration towards S1P by Jurkat cells expressing EDG1 and EDG3. Several clones are compared for EDG1 and EDG3.

FIG. 3 depicts an example of a high throughput migration assay of the invention.

FIG. 4 is a graph depicting robust migration of EDG1 #15 clone in response to S1P induction detected in a 96-well format.

FIG. 5A is a graph depicting migration of EDG1 #15 clone in response to increasing S1P concentration.

FIG. 5B is a graph depicting migration of EDG1 #15 clone over six hours. Maximum migration is at 4 to 5 hours.

FIG. 6 is a graph depicting titration of S1P concentration with EDG1 #15 clone.

FIG. 7A is a graph depicting inhibition of migration of EDG1 #15 clone by FTY720, an immunosuppressant.

FIG. 7B is a graph depicting inhibition of migration of EDG1 #15 clone versus EDG3 #1 clone by FTY720, an immunosuppressant. FTY720 inhibits the migration of EDG1#15 clone but not EDG3 #1 clone.

FIG. 8A is a graph depicting stability of EDG3 clones (EDG#1 and EDG#3) up to 60 days after sorting.

FIG. 8B is a graph depicting migration of EDG3#1 clones over 5 hours.

FIG. 9 is a graph depicting titration of S1P concentration with EDG3 #1 clone.

FIG. 10A is a graph depicting inhibition of EDG3 #1 clone migration by suramin.

FIG. 10B is a graph depicting differential inhibition of EDG1 and EDG3 migration by suramin.

### DETAILED DESCRIPTION OF THE INVENTION

The present invention is based, at least in part, on the development of assays for the identification of compounds which modulate cell migration or activation, e.g., lymphocyte or endothelial cell migration or activation. Cells for use in the assays overexpress migration molecules and their use in methods of the invention enables one to employ high-throughput drug screening protocols. High-throughput screening (HTS) methods, compositions, and kits for performing the assays are also provided.

Using the instant assays, it has been found that overexpression of EDG1 in T or B cells inhibits anti-T cell receptor (TCR) or anti-IgM-induced CD69 induction, while overexpression of EDG3 in T cells enhances anti-TCR-induced CD69 induction. Furthermore, overexpression of EDG1 and EDG3 in T lymphoid cells enhance the migration activity in response to a ligand, e.g., S1P.

In general, the subject assays are performed by contacting a cell which overexpresses (e.g., stably or transiently overexpresses) a migration molecule with a test agent and a migration molecule ligand, and measuring migration of the cell towards the ligand. In one embodiment, cell migration is mediated by one or more migration molecule, e.g., an EDG molecule (e.g., EDG1 and EDG3), a selectin molecule, an integrin molecule, a cadherin molecule, certain members of the immunoglobulin superfamily of molecules or a chemokine receptor molecule. In another embodiment, cells which stably overexpress the migration molecule are specifically selected for use in the high throughput screening assays of the invention. In another embodiment, cells are detected by fluorescence activated cell sorting (FACS). In still another embodiment, the assay is carried out in a high throughput format, e.g., in a 96-well format. The screening assays of the invention may or may not be automated.

The present invention is also based, at least in part, on the generation of cells, e.g., Jurkat cells, that are capable of overexpressing migration molecules, for example, EDG molecules (e.g., EDG1 and EDG3), selectin molecules, integrin molecules, cadherin molecules, certain members of the immunoglobulin superfamily of molecules or chemokine receptor molecules which may be used in the high throughput assays of the invention. In particular, the present invention includes cells, e.g., T lymphoid cells, which overexpress EDG1 and EDG3, respectively. Both cell lines exhibit enhanced migration activity toward a ligand, S1P. EDG1 expression is regulated by tetracycline. In the presence of doxycycline, the EDG1-mediated migration is abolished.

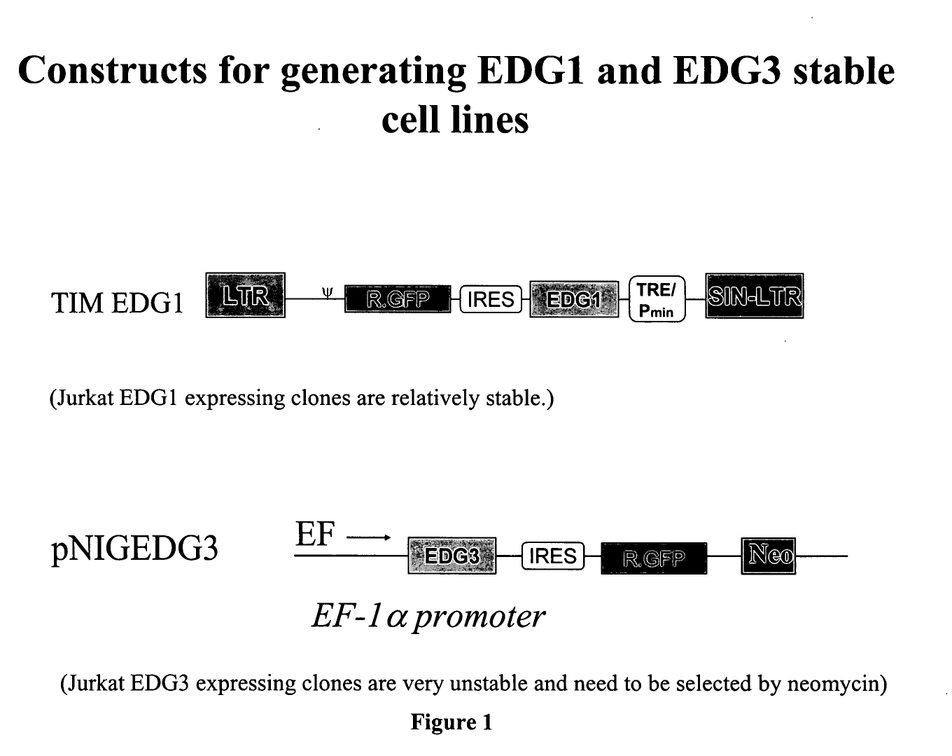

The present invention also includes vectors, e.g., retroviral vectors, which may be used to transform cells such that the cells overexpress the desired migration molecule. Preferably, the cells stably overexpress the migration molecule(s). Examples of constructs which may be used to transform cells for generating EDG1 and EDG3 stable cell lines includes those set forth in FIG. 1.

Modulators of cell migration which are identified using the methods of the invention may be used for the treatment and/or prevention of cell migration-associated diseases or disorders.

Various aspects of the invention are described in further detail in the following subsections:

I. Definitions

“Cell migration” refers to migration of cells via the blood stream, lymphatic vessels, and by penetration of capillary walls (see, e.g., Paul, Immunology (3rd ed., 1993) (Chapters 4 and 6)). Exemplary cells capable of cell migration include, but are not limited to, immune cells, B cells, T cells, or endothelial cells. Further examples of such cells are provided throughout the specification. Cell migration includes whole-cell locomotion and the regulation of the cell shape and extracellular attachment. Cell migration is crucial for several normal and pathological processes, including: cell and tissue development, wound healing, inflammation, immune response, and metastases of tumors.

Migration can be effected by migration molecules expressed by the cells. For example, EDG proteins, e.g., EDG-1 and EDG-3, participate in the process of lymphocyte migration via ligand binding to and or activation of the EDG protein (e.g., using SPP (sphingosine-1-phosphate, also known as S1P) or LPA (lysophosphatidic acid) or analogs thereof, and/or cytokines). SPP and LPA are present in serum and are produced by a number of cells, including platelets and fibroblasts. Ligand-induced lymphocyte migration can be measured using the assay described herein, in which lymphocytes migrate toward the ligand from an upper to a lower chamber. The sphingolipid analog compound 2-amino-2(2-[4-octylphenylethyl)-1,3-propanediol hydrochloride and analogs thereof inhibit such migration. The C-terminus of EDG-1 appears to be involved in migration. Such domains (e.g., the cytoplasmic tail of EDG-1) can be used in high throughput binding assays for compounds that modulate lymphocyte migration.

“Lymphocyte activation” refers to the process of stimulating quiescent (G₀ phase of cell cycle), mature B and T cells by encounter with antigen, either directly or indirectly (e.g., via a helper cell and antigen presenting cells as well as via direct antigen contact with a cell surface molecule of the lymphocyte). Characteristics of activation can include, e.g., increase in cell surface markers such as CD69, entry into the G1 phase of the cell cycle, cytokine production, and proliferation (see, e.g., Paul, Immunology (3rd ed., 1993) (Chapters 13 and 14)).

Cells can migrate in response to the binding of migration molecules to the ligands they recognize. In vitro, cellular migration is often measured using chemotaxis or haptotaxis assays. In chemotaxis, diffusible chemical signals can cause cells to migrate preferentially in a given direction, typically up the gradient of the factor. Alternatively, in haptotaxis bound molecules, either on the surfaces of adjacent cells or in the extracellular matrix, can provide adhesive gradients that guide cell movements in a preferred direction.

The term “migration molecule,” “migration polypeptide” or “migration nucleic acid” refers to any molecule which is expressed on a cell surface, e.g., B lymphocyte, T lymphocyte, or endothelial cell surface, and which is involved in or mediates the migration or recruitment of a cell, e.g., a lymphocyte or endothelial cell. Examples of migration molecules include EDG molecules, including, but not limited to EDG1 and EDG3, and chemokine receptors, e.g., CCR1, CCR2, CCR3, CCR4, CCR5, CCR6, CCR7, CCR8, CCR9, CCR10, CCR11, CXCR1, CXCR2, CXCR3, CXCR4, CXCR5, CX3CR1, and XCR1.

Leukocyte recruitment involves a cascade of cellular events including initial attachment, rolling, weak and firm adhesion, diapedesis, transendothelial migration and chemotaxis. At least four families of cell adhesion molecules are involved in the interactions of leukocytes with endothelial cells. These families of molecules include: selectins and their glycoprotein ligands, integrins and their counter-receptors, and the immunoglobulin superfamily of cell adhesion molecules.

In one embodiment, migration molecules is a cellular adhesion molecules. The term “adhesion molecule” refers to any molecule which is expressed on the cell surface molecule and mediates or is involved in cell-to-cell binding, e.g., endothelial cell or leukocyte cell binding, or binding of cells to the extracellular matrix. Adhesion molecules are integral membrane proteins that have cytoplasmic, transmembrane and extracellular domains.

Adhesion molecules include, for example, selectins, e.g., L-selectin, E-selectin, and P-selectin, integrins, e.g., α1β1, α2β1, α3β1, α4β1, α5β1, α6β1, α7β1, α8β1 (VLA-8), α9β1β1, αVβ1, αLβ2, αMβ2, αXβ2, αIIβ3, α6β3, α6β4, αvβ3, αVβ5, αVβ6, αVβ8, α4β7, αIELPβ7, and α11, cadherins, e.g., Cadherin E (1), Cadherin N (2), Cadherin BR (12), Cadherin P (3), Cadherin R (4), Cadherin M (15), Cadherin VE (5) (CD144), Cadherin T & H (13), Cadherin OB (11), Cadherin K (6), Cadherin 7, Cadherin 8, Cadherin KSP (16), Cadherin LI (17), Cadherin 18, Cadherin, Fibroblast 1 (19), Cadherin Fibroblast 2 (20), Cadherin Fibroblast 3 (21), Cadherin 23, Desmocollin 1, Desmocollin 2, Desmoglein 1, Desmoglein 2, Desmoglein 3, and Protocadherin 1, 2, 3, 7, 8, and 9, and members of the immunoglobulin superfamily which function as adhesion molecules, e.g., Inter-Cellular Adhesion Molecule-1 (I-CAM-1) (CD54), Inter-Cellular Adhesion Molecule-2 (I-CAM-2) (CD102), Inter-Cellular Adhesion Molecule-3 (I-CAM-3) (CD50), and Vascular-Cell Adhesion Molecule (V-CAM), ALCAM (CD166), Basigin (CD147), BL-CAM (CD22), CD44, Lymphocyte function antigen-2 (LFA-2) (CD2), LFA-3 (CD 58), Major histocompatibility complex (MHC) molecules, MAdCAM-1, PECAM (CD31). Other immunoglobulin superfamily molecules which are adhesion molecules are expressed predominately in nervous tissue and are referred to as neural cell adhesion molecules (N-CAMs).

Any one or more migration molecule may be used in the methods of the invention to identify modulators of cell migration which is mediated by a migration molecule. The amino acid sequence of migration molecules for use in the invention are known in the art or can be readily determined by one of ordinary skill in the art. It will be understood that variants of these molecules (i.e., molecules differing in amino acid sequence from reference amino acid sequence, but retaining the same activity may also be used in the methods of the invention. Reference sequences can be obtained, e.g., from a database such as GenBank.

In a preferred embodiment, the terms “EDG migration polypeptide or fragment thereof,” or a “nucleic acid encoding an EDG migration polypeptide or fragment thereof” refer to nucleic acids and polypeptide polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to an amino acid sequence encoded by an EDG nucleic acid or amino acid sequence of a migration molecule, e.g., an EDG protein, e.g., EDG-1, 3, 5, 6, 8, or EDG-2, 4, and 7; (2) specifically bind to antibodies, e.g., polyclonal antibodies, raised against an immunogen comprising an amino acid sequence of a migration molecule, e.g., an EDG protein, e.g., EDG-1, 3, 5, 6, 8, or EDG-2, 4, and 7, immunogenic fragments thereof, and conservatively modified variants thereof; (3) specifically hybridize under stringent hybridization conditions to an anti-sense strand corresponding to a nucleic acid sequence encoding a migration molecule, e.g., an EDG protein, e.g., EDG-1, 3, 5, 6, 8, or EDG-2, 4, and 7, and conservatively modified variants thereof; (4) have a nucleic acid sequence that has greater than about 60% sequence identity, 65%, 70%, 75%, 80%, 85%, 90%, preferably 91%, 92%, 93%, 94%, 95%, 96%, 97%, 98% or 99%, or higher nucleotide sequence identity, preferably over a region of at least about 25, 50, 100, 200, 500, 1000, or more nucleotides, to a migration molecule, e.g., an EDG nucleic acid, e.g., EDG-1, 3, 5, 6, 8, or EDG-2, 4, and 7. EDG molecules are described in U.S. Application No. 20020155512, the contents of which are incorporated herein by reference.

The term “recombinant” when used with reference, e.g., to a cell, or nucleic acid, protein, or vector, indicates that the cell, nucleic acid, protein or vector, has been modified by the introduction of an exogenous nucleic acid or protein or the alteration of a native nucleic acid or protein, or that the cell is derived from a cell so modified. Exogenous nucleic acid molecules can be derived from a different species or from the same species. Thus, for example, recombinant cells express genes that are not found within the wild-type (non-recombinant) form of the cell or express native genes that are otherwise abnormally expressed, e.g., overexpressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. For example, a promoter that normally encodes a different gene or from a different organism operably linked to a gene encoding a migration molecule. Similarly, a heterologous protein indicates that the protein comprises two or more subsequences that are not found in the same relationship to each other in nature (e.g., a fusion protein).

The term “overexpression” as used herein, refers to the expression of a polypeptide, e.g., a migration molecule as described herein, by a cell, at a level which is greater than the normal level of expression of the polypeptide in a cell which normally expresses the polypeptide. For example, expression of the polypeptide may by 10%, 20%, 30%, 40%, 50%, 60%, 70, 80%, 90%, 100%, or more as compared to expression of the polypeptide in a wild-type cell which normally expresses the polypeptide. Mutants variants, or analogs of the polypeptide of interest may be overexpressed.

As used herein, the term “transient” expression refers to expression of exogenous nucleic acid molecule(s) which are separate from the chromosomes of the cell. Transient expression generally reaches its maximum 2-3 days after introduction of the exogenous nucleic acid and subsequently declines.

As used here, the term “stable” expression refers to expression of exogenous nucleic acid molecule(s) which are part of the chromosomes of the cell. In general, vectors for stable expression of genes include one or more selection marker.

As used herein, the term “reporter gene” or “selection gene” or “resistance gene” is meant a gene that by its presence in a cell (e.g., upon expression) allows the cell to be distinguished from a cell that does not contain the reporter gene. Reporter genes can be classified into several different types, including detection genes, survival genes, death genes, cell cycle genes, cellular biosensors, proteins producing a dominant cellular phenotype, and conditional gene products. As is more fully outlined below, additional components, such as substrates, ligands, etc., may be additionally added to allow selection or sorting on the basis of the reporter gene.

“Inhibitors,” “activators,” and “modulators” of a migration molecule are used to refer to activating, inhibitory, or modulating molecules identified using the subject in vitro or in vivo assays. Inhibitors are compounds that, e.g., bind to, partially or totally block activity, decrease, prevent, delay activation, inactivate, desensitize, or down regulate the activity or expression of a migration molecule, e.g., antagonists. “Activators” are compounds that increase, open, activate, facilitate, enhance activation, sensitize, agonize, or up regulate migration molecule activity. Inhibitors, activators, or modulators also include genetically modified versions of migration molecules, e.g., versions with altered activity, as well as naturally occurring and synthetic ligands, antagonists, agonists, peptides, cyclic peptides, nucleic acids, antibodies, antisense molecules, ribozymes, small organic molecules and the like. Such assays for inhibitors and activators include, e.g., expressing a migration molecule in vitro, in cells, cell extracts, or cell membranes, applying putative modulator compounds, and then determining the functional effects on activity, as described above.

Samples or assays comprising migration molecules that are treated with a potential activator, inhibitor, or modulator are compared to control samples without the inhibitor, activator, or modulator to examine the extent of activation or migration modulation. Control samples (untreated with inhibitors) are assigned a relative protein activity value of 100%. Inhibition of a migration molecule is achieved when the activity value relative to the control is about 80%, preferably 50%, more preferably 25-0%. Activation of a migration molecule is achieved when the activity value relative to the control (untreated with activators) is 110%, more preferably 150%, more preferably 200-500% (i.e., two to five fold higher relative to the control), more preferably 1000-3000% higher.

The term “test agent” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid (e.g., a sphingolipid), fatty acid, polynucleotide, oligonucleotide, etc., which is employed in the assays of the invention and assayed for its ability to influence cell migration. The test agent can be in the form of a library of test agents, such as a combinatorial or randomized library that provides a sufficient range of diversity. Test agents are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test agent (called a “lead compound”) with some desirable property or activity, e.g., inhibiting activity, creating variants of the lead compound, and evaluating the property and activity of those variant compounds.

More than one compound, e.g., a plurality of compounds, can be tested at the same time for their ability to modulate cell migration. In one embodiment, the term “screening assay” preferably refers to assays which test the ability of a plurality of compounds to influence the readout of choice rather than to tests which test the ability of one compound to influence a readout. Preferably, the subject assays identify compounds not previously known to have the effect that is being screened for. In one embodiment, high throughput screening may be used to assay for the activity of a compound.

In preferred embodiments of the invention, high throughput screening (HTS) methods are employed to measure cellular migration. High throughput molecular screening (HTS) is the automated, simultaneous testing of thousands of distinct chemical compounds in models of biological mechanisms or disease.

Known modulators of migration can be used as controls in the instant assays. One such molecule “FTY720” is a chemical molecule of the formula 2-amino-2(2-[4-octylphenylethyl)-1,3-propanediol hydrochloride. FTY720 is a sphingolipid analog. FTY720 and analogs thereof are useful for inhibiting EDG-1 and EDG family mediated lymphocyte migration. FTY720 and analogs thereof are designed and made according to methods known to those of skill in the art (see, e.g., U.S. Pat. No. 6,004,565, U.S. Pat. No. 5,604,229, and PCT application PCT/JP95/01654, and Fujita et al., J. Antibiotics 47:216-224 (1994)).

“Biological sample” include sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate, e.g., chimpanzee or human; cow; dog; cat; a rodent, e.g., guinea pig, rat, mouse; rabbit; or a bird; reptile; or fish.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 seconds to 2 minutes, and an extension phase of about 72° C. for 1-2 minutes. Protocols and guidelines for low and high stringency amplification reactions are provided, e.g., in Innis et al. (1990) PCR Protocols, A Guide to Methods and Applications, Academic Press, Inc. N.Y.).

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to EDG protein, polymorphic variants, alleles, orthologs, and conservatively modified variants, or splice variants, or portions thereof, can be selected to obtain only those polyclonal antibodies that are specifically immunoreactive with EDG proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

By “therapeutically effective dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

The present invention also includes methods of using the modulators identified by the methods of the invention to treat or prevent “cell migration-associated diseases or disorders.” As used herein, a “cell migration-associated disease or disorder” includes, without limitation, a disease state which is marked by either an excess or a deficit of cell activation or migration, e.g., T cell, B cell, or endothelial cell migration or activation. For example, cell migration-associated diseases or disorders include, but are not limited to, disorders that would benefit from modulation of angiogenesis, referred to herein as “angiogenic diseases or disorders.” An angiogenic disease or disorder includes a disease or disorder characterized by aberrantly regulated angiogenesis. Angiogenesis is the sprouting of new blood vessels, e.g., capillaries, vessels, and veins from pre-existing vessels characterized by expansion of the endothelium by proliferation, migration and remodeling. Angiogenesis is a multistep process, which involves retraction of pericytes from the abluminal surface of the capillary, release of proteases from the activated endothelial cells, degradation of the extracellular matrix (ECM) surrounding the pre-existing vessels, endothelial cell migration toward an angiogenic stimulus and their proliferation, formation of tube-like structures, fusion of the formed vessels and initiation of blood flow. New blood vessels can develop from the walls of existing small vessels by the outgrowth of endothelial cells.

Angiogenesis is also involved in tumor growth as it provides tumors with the blood supply necessary for tumor cell survival and proliferation (growth). Accordingly an example of an angiogenic disease includes solid tumor growth and metastasis, e.g., ovarian, lung, cervical, breast, endometrial, uterine, hepatic, gastrointestinal, prostate, colorectal, and brain tumors. As used herein, a “tumor” includes a normal benign or malignant mass of tissue.

Other angiogenic diseases or disorders include, for example, psoriasis, endometriosis, Grave's disease, ischemic disease (e.g., atherosclerosis), atherosclerosis, and chronic inflammatory diseases (e.g., rheumatoid arthritis), and some types of eye disorders, including diabetic retinopathy, macular degeneration, neovascular glaucoma, inflammatory diseases and ocular tumors (e.g., retinoblastoma), retrolental fibroplasia, uveitis, eye diseases associated with choroidal neovascularization and eye diseases which are associated with iris neovascularization. The methods of the invention may be used to identify modulators of angiogenesis, e.g., anti-angiogenesis compounds.

Migration molecules play a critical role in angiogenesis via the mediation of endothelial cell adhesion, e.g., adhesion with other endothelial cells and with the extracellular matrix, and cellular migration (Zoltan, et al. (1999) Trends in Glycoscience and Glycobiology 11(56):73-93, the contents of which are incorporated herein by reference). For example, E-selectin, VCAM-1, CD31, and some integrins have been shown to facilitate capillary formation in vitro and in vivo and a number of migration molecules have been shown to be differentially expressed in angiogenic diseases, e.g., cancer and rheumatoid arthritis. Therefore, migration molecules play a role in the pathogenesis of angiogenic diseases or disorders.

Additional cell migration-associated diseases or disorders include, but are not limited to, thrombosis, immune disease, autoimmune disease, myocardial infarction, bacterial or viral infection, metastatic conditions, inflammatory disorders such as arthritis, gout, uveitis, acute respiratory distress syndrome, asthma, emphysema, delayed type hypersensitivity reaction, systemic lupus erythematosus, thermal injury such as burns or frostbite, autoimmune thyroiditis, experimental allergic encephalomyelitis, multiple sclerosis, multiple organ injury syndrome secondary to trauma, diabetes, Reynaud's syndrome, neutrophilic dermatosis (Sweet's syndrome), inflammatory bowel disease, Grave's disease, glomerulonephritis, gingivitis, periodontitis, hemolytic uremic syndrome, ulcerative colitis, Crohn's disease, necrotizing enterocolitis, granulocyte transfusion associated syndrome, cytokine-induced toxicity, organ transplant rejection, and the like.

Leukocyte extravasation is crucial for appropriate and effective immune response. Neutrophils normally exist in a resting state as they circulate though the body. However, upon interaction with small molecules known as chemoattractants, they rapidly respond with endothelial adhesion followed by emigration from the vasculature and chemotaxis to the site of inflammation. Once at the site of inflammation, neutrophils respond with phagocytosis, superoxide generation, and the release of degradative enzymes. Therefore, modulation of leukocyte migration results in modulation of immune and inflammatory response and would be an effective modulator of autoimmune-related diseases or disorders and/or inflammatory diseases and disorders. Furthermore, pathological states for which it may be desirable to increase lymphocyte activation or migration include HIV infection that results in immunocompromise, cancer, and infectious disease such as viral, fungal, protozoal, and bacterial infections. Different compounds may be used to modulate cell activation and migration, or the same compound may be used to modulate cell activation and migration.

The term “retroviral vectors” as used herein includes vectors used to introduce the nucleic acids of the present invention into a host in the form of an RNA viral particle, as is generally outlined in PCT US 97/01019 and PCT US 97/01048, both of which are incorporated by reference.

As used herein, a “self-inactivating long terminal repeat (SIN-LTR)” is a retroviral long terminal repeat region which comprises a deletion in the U3 region of the 3′LTR. During reverse transcription, this deletion is transferred to the 5′LTR of the proviral DNA. If enough sequence is eliminated to abolish transcriptional activity of the LTR, the production of full-length vector RNA in the host cell is abolished.

As used herein a “transcriptional response element (TRE)” is a cell specific response element, which may be used with an adenovirus gene that is essential for propagation, so that replication competence is only achievable in the target cell, and/or with a transgene for changing the phenotype of the target cell.

As used herein an “internal ribosome entry site (IRES)” is a site in a nucleic acid molecule which allows efficient internal initiation of translation ensuring coordinate expression of several genes. IRES sites can be used as linkers which may be used to link a first nucleic acid to the 5′ end or the 3′ end of a second nucleic acid. The expression products of such a vector include a fusion nucleic acid and two separate polypeptides translated from the fusion nucleic acid.

As used herein the term “fusion nucleic acid” refers to a plurality of nucleic acid components that are joined together, either directly or indirectly. As will be appreciated by those in the art, in some embodiments the sequences described herein may be DNA, for example when extrachromosomal plasmids are used, or RNA when retroviral vectors are used. In some embodiments, the sequences are directly linked together without any linking sequences while in other embodiments linkers such as restriction endonuclease cloning sites, linkers encoding flexible amino acids, such as glycine or serine linkers such as known in the art, are used, as further discussed below. In one embodiment, a fusion nucleic acid may encode two distinct proteins, e.g., a test agent and a migration molecule.

As used herein an “elongation-factor 1α (EF-1α) promoter” is derived from the EF-1α gene encoding elongation factor-1α, which is an enzyme which catalyzes the GTP-dependent binding of aminoacyl-tRNA to ribosomes. EF-1α is one of the most abundant proteins in eukaryotic cells and is expressed in almost all kinds of mammalian cells. The promoter of this ‘housekeeping’ gene exhibits a strong activity, yields persistent expression of the transgene in vivo.

Various aspects of the invention are described in further detail in the following subsections:

II. Screening Assays

The present invention provides methods (also referred to herein as “screening assays”) for identifying modulators, i.e., candidate or test agents (e.g., peptidomimetics, small molecules or other drugs) which modulate cell migration or activation, e.g., lymphocyte or endothelial cell migration or activation.

The assays can be used to identify agents that modulate the function of migration molecules, e.g., EDG molecules, e.g., EDG1 and EDG3, selectin, integrin, cadherin, certain members of the immunoglobulin superfamily of molecules or chemokine receptor molecules. For example, such agents may interact with one or more migration molecule or nucleic acid molecule which regulates expression of a migration molecule (e.g., to inhibit or enhance its activity or expression). The function of the migration molecule can be affected at any level, including transcription, protein expression, protein localization, and/or cellular activity. The subject assays can also be used to identify, e.g., agents that alter the interaction of the migration molecule with a binding partner, substrate, or cofactors, or modulate, e.g., increase or decrease, the stability of such interaction.

In one embodiment, the screening assays of the invention are high throughput or ultra high throughput (e.g., Fernandes, P. B., Curr Opin Chem Biol. 1998 2:597; Sundberg, S A, Curr Opin Biotechnol. 2000, 11:47). For example, the screening assays of the invention a may be carried out in a multi-well format, for example, a 96-well, 384-well format, or 1,536-well format, and are suitable for automation. In the high throughput assays of the invention, it is possible to screen up to several thousand different modulators or ligands in a single day. In particular, each well of a microtiter plate can be used to run a separate assay against a selected test agent, or, if concentration or incubation time effects are to be observed, every 5-10 wells can test a single modulator. Thus, a single standard microtiter plate can assay about 100 (e.g., 96) modulators. If 1,536 well plates are used, then a single plate can easily assay from about 100-about 1500 different compounds. It is possible to assay many plates per day; assay screens for up to about 6,000, 20,000, 50,000, or more than 100,000 different compounds are possible using the assays of the invention.

In one embodiment, a high throughput binding assay is performed in which the migration molecule, or fragment thereof, is contacted with a test agent and a ligand and incubated for a suitable amount of time. In one embodiment, the test agent is bound to a solid support. In another embodiment, the migration molecule is bound to a solid support. In one embodiment, the test agent is bound to a support. In another embodiment, the test agent is contacted with a cell expressing a migration molecule. In another embodiment, the test agent is expressed by a cell. A wide variety of modulators can be used, as described below, including small organic molecules, peptides, and antibodies. In one embodiment, the cell stably overexpresses a migration molecule. In another embodiment, the cell transiently overexpresses a migration molecule. The cell may be, for example, an immune cell, e.g., a lymphocyte, or an endothelial cell. In one embodiment, the cell is a Jurkat cell.

The assays of the invention may be chemotactic or haptotactic. In a chemotactic assay, diffusible chemical signals or chemoattractants can cause cells to migrate preferentially in a given direction, typically up the gradient of the factor. In haptotactic assays, a molecule recognized by a migration molecule can be attached to a solid support or expressed by a cell. For example, cells may migrate from one vessel to another vessel, for example, through a membrane, e.g., a microporous membrane, towards a chemoattractant ligand such as S1P. Alternatively, in a haptotaxis assay, bound molecules, either on the surfaces of adjacent cells or in an extracellular matrix provide adhesive gradients that guide cell movements in a preferred direction.

In setting up the subject assays, the components may be added in any order. For example, in one embodiment, the cell expressing the migration molecule and its ligand are contacted prior to addition of the test agent. In another embodiment, the test agent is added prior to addition of the cell expressing the migration molecule or the ligand. In a preferred embodiment, the test agent is added together with a ligand, e.g., S1P, in a bottom receiver plate and cells are placed in an upper filter plate, where, for example, the receiver plate and the upper plate are separated by a membrane. The cells migrate to the receiver plate. In one embodiment, the cells are stained with a fluorescent dye. The cells may then be detected by a fluorescence reader, e.g., a fluorescence plate reader. Interference with binding, either of the test agent or of the known ligand, is determined. In another embodiment, either the test agent or the known ligand is labeled.

Cell migration may be determined through direct measurements of migration. For example, cell migration may be measured by labeling cells either before or after migration, and obtaining a readout based on the measurement of the location of the labeled cells. For example, cells may be labeled using a fluorescent label, e.g., CyQUANT™ (Molecular Probes™)), and migration may be determined using a fluorescence plate reader using, for example, a 480/520 nm filter set. Other labels that may be used in the methods of the invention include radioactive labels, e.g., ³²P, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

In another embodiment, indirect measurements of migration can be made. For example, the expression of a molecule associated with cell migration (e.g., the expression of which is decreased or increased in migrating cells) can be measured.

In some cases, the binding of the candidate modulator is determined through the use of competitive binding assays, where interference with binding of a known ligand is measured in the presence of a test agent.

Compounds that modulate cell migration identified using the assays described herein can be useful for treating a subject that would benefit from the modulation of the migration molecule, e.g., a subject having or at risk for a cell migration-associated disease or disorder.

In one embodiment, the subject assays can be used as secondary assays can be used to confirm that the modulating agent affects the migration molecule in a specific manner. For example, compounds identified in a primary screening assay can be used in a secondary screening assay to determine whether the compound affects cell migration. In another embodiment, a compound identified in one of the subject assays can be tested in a secondary assay, e.g., in an animal model of a cell migration-associated disease or disorder to confirm its activity. Accordingly, in another aspect, the invention pertains to a combination of two or more of the assays described herein.

Moreover, a modulator of cell migration identified as described herein (e.g., a small molecule) may be used in an animal model to determine the efficacy, toxicity, or side effects of treatment with such a modulator. Alternatively, a modulator identified as described herein may be used in an animal model to determine the mechanism of action of such a modulator.

Indirect measurements of cell migration may also be used in the instant application, e.g., as primary screens for modulators of migration, or to confirm the activity of a modulator identified through direct measurement of cell migration. A wide variety of assays can be used to identify migration molecule-modulator binding, including labeled protein-protein binding assays, electrophoretic mobility shifts, immunoassays, enzymatic assays such as phosphorylation assays, and the like.

In another embodiment, the migration molecule is expressed in a cell, and functional, e.g., physical and chemical or phenotypic, changes are assayed to identify cell migration and activation modulators. Cells expressing migration molecules can also be used in binding assays. Any suitable functional effect can be measured, as described herein. For example, ligand binding, cell surface marker expression, cellular proliferation, apoptosis, cytokine production, and GPCR signal transduction, e.g., changes in intracellular Ca²⁺ levels, are all suitable assays to identify test agents using a cell based system. Suitable cells for such cell based assays include both primary lymphocytes and cell lines, as described herein. The migration molecule can be naturally occurring or recombinant. Also, as described above, fragments of the migration molecule or fusion protein with cell migration or activation activity, e.g., G protein coupled receptor (GPCR) can be used in cell based assays. For example, the extracellular domain of an EDG protein can be fused to the transmembrane and/or cytoplasmic domain of a heterologous protein, preferably a heterologous GPCR. Such a chimeric GPCR would have GPCR activity and could be used in cell based assays of the invention. In another embodiment, a domain of the migration protein, such as the extracellular or cytoplasmic domain, is used in the cell-based assays of the invention.

As described above, in one embodiment, cell migration is measured by contacting cells comprising a target with a test agent. Modulation of T cell migration can be measured by screening for expression of migration molecules, using fluorescent antibodies and FACS sorting. In another embodiment, migration is measured by observing cell migration from an upper to a lower chamber containing a migration molecule ligand such as, for example, SPP or a chemokine.

In another embodiment, cellular migration can be measured using 3H-thymidine incorporation or dye inclusion. In another embodiment, cellular migration molecule levels are determined by measuring the level of protein or mRNA. The level of the migration molecules are measured using immunoassays such as western blotting, ELISA and the like with an antibody that selectively binds to the migration molecule or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, the migration molecule expression can be measured using a reporter gene system. Such a system can be devised using a migration molecule protein promoter operably linked to a reporter gene such as chloramphenicol acetyltransferase, firefly luciferase, bacterial luciferase, β-galactosidase and alkaline phosphatase. Furthermore, the protein of interest can be used as an indirect reporter via attachment to a second reporter such as red or green fluorescent protein (see, e.g., Mistili & Spector, Nature Biotechnology 15:961-964 (1997)). The reporter construct is typically transfected into a cell. After treatment with a test agent, the amount of reporter gene transcription, translation, or activity is measured according to standard techniques known to those of skill in the art.

Recombinant expression vectors that may be used for expression of polypeptides are known in the art. For example, the cDNA is first introduced into a recombinant expression vector using standard molecular biology techniques. A cDNA can be obtained, for example, by amplification using the polymerase chain reaction (PCR) or by screening an appropriate cDNA library.

When used in mammalian cells, the expression vector's control functions are often provided by viral regulatory elements. For example, commonly used promoters are derived from polyoma virus, adenovirus, cytomegalovirus and Simian Virus 40. Non-limiting examples of mammalian expression vectors include pCDM8 (Seed, B., (1987) Nature 329:840) and pMT2PC (Kaufinan, et al. (1987), EMBO J. 6:187-195). A variety of mammalian expression vectors carrying different regulatory sequences are commercially available. For constitutive expression of the nucleic acid in a mammalian host cell, a preferred regulatory element is the cytomegalovirus promoter/enhancer. Moreover, inducible regulatory systems for use in mammalian cells are known in the art, for example systems in which gene expression is regulated by heavy metal ions (see e.g., Mayo, et al. (1982) Cell 29:99-108; Brinster, et al. (1982) Nature 296:39-42; Searle, et al. (1985) Mol. Cell. Biol. 5:1480-1489), heat shock (see e.g., Nouer, et al. (1991) in Heat Shock Response, e.d. Nouer, L., CRC, Boca Raton, Fla., pp 167-220), hormones (see e.g., Lee, et al. (1981) Nature 294:228-232; Hynes, et al. (1981) Proc. Natl. Acad. Sci., USA 78:2038-2042; Klock, et al. (1987) Nature 329:734-736; Israel & Kaufman (1989) Nucl. Acids Res. 17:2589-2604; and PCT Publication No. WO 93/23431), FK506-related molecules (see e.g., PCT Publication No. WO 94/18317) or tetracyclines (Gossen, M. and Bujard, H. (1992) Proc. Natl. Acad. Sci., USA 89:5547-5551; Gossen, M. et al. (1995) Science 268:1766-1769; PCT Publication No. WO 94/29442; and PCT Publication No. WO 96/01313). Still further, many tissue-specific regulatory sequences are known in the art, including the albumin promoter (liver-specific; Pinkert, et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji, et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci., USA 86:5473-5477), pancreas-specific promoters (Edlund, et al. (1985) Science 230:912-916) and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Additional methods of introducing nucleic acid molecules into cells are provided below.

A. Migration Molecules and Ligands Thereof

In one embodiment, a migration molecule of the invention is an EDG molecule. EDG proteins often have GPCR activity, e.g., the ability to transduce a signal via a G protein in response to extracellular ligand binding. For example, EDG-1 is coupled to G₁, a pertussis toxin-sensitive G protein. Binding of SPP to EDG-1 results in inhibition of adenylate cyclase and activation of MAPK (both G₁-mediated) as well as upregulation of P- and E-cadherin expression and Rho-dependent morphogenesis. There are eight members of the EDG family (EDG1, EDG2, EDG3, EDG4, EDG5, EDG6, EDG7, and EDG8). They are all G protein coupled receptors are glycoproteins that share certain structural similarities (see, e.g., Gilman, Ann. Rev. Biochem. 56:615-649 (1987), Strader et al., The FASEB J. 3:1825-1832 (1989), Kobilka et al., Nature 329:75-79 (1985), and Young et al., Cell 45:711-719 (1986)). For example, G protein coupled receptors have an extracellular domain, seven hydrophobic stretches of about 20-25 amino acids in length interspersed with eight hydrophilic regions (collectively known as the transmembrane domain), and a cytoplasmic tail. Each of the seven hydrophobic regions forms a transmembrane alpha helix, with the intervening hydrophilic regions forming alternatively intracellular and extracellular loops. The third cytosolic loop between transmembrane domains five and six is involved in G-protein interaction. These transmembrane hydrophobic domains, hydrophilic loop domains, extracellular domains, and cytoplasmic tail domains can be structurally identified using methods known to those of skill in the art, such as sequence analysis programs that identify hydrophobic and hydrophilic domains (see, e.g., Kyte & Doolittle, J. Mol. Biol. 157:105-132 (1982)). Such domains are useful for making chimeric proteins and for in vitro assays of the invention (see, e.g., WO 94/05695 and U.S. Pat. No. 5,508,384). Such domains are also considered “fragments” of EDG proteins, and as such are useful in the assays of the invention.

The Unigene number for EDG-1 is Hs. 154210, and GenBank accession numbers for exemplary nucleotide and amino acids sequences are NM._{—}001400, XM_{—}001499, NP_{—}001391, XP_{—}00149, AAC51905, AAF43420, and AAA52336. The chromosomal location is Chr 1p21. The OMIM reference number for EDG-1 is 601974. EDG-1 is expressed in, e.g., in endothelial cells, vascular smooth muscle cells, fibroblasts, melanocytes and cells of epithelioid origin (see, e.g., Hla & Maciag, J. Biol. Chem. 265:9308-9313 (1990); Hobson et al., Science 291:1800-1803 (2001); and Lee et al., Science 279:1552-1555 (1998)).

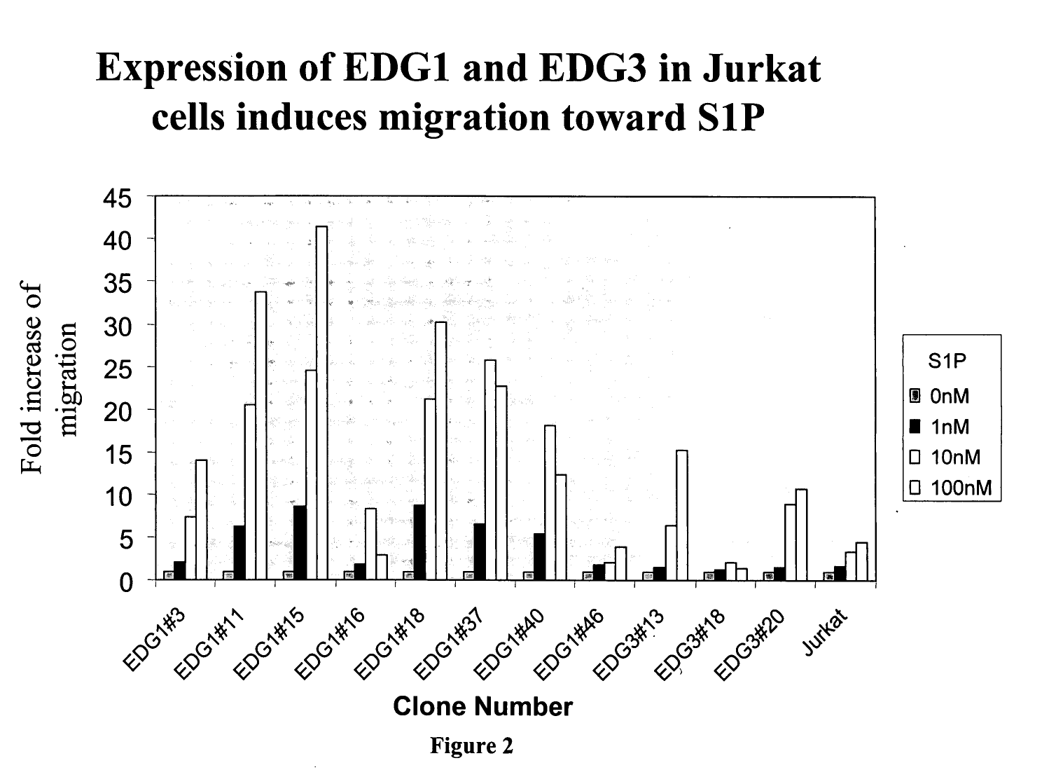

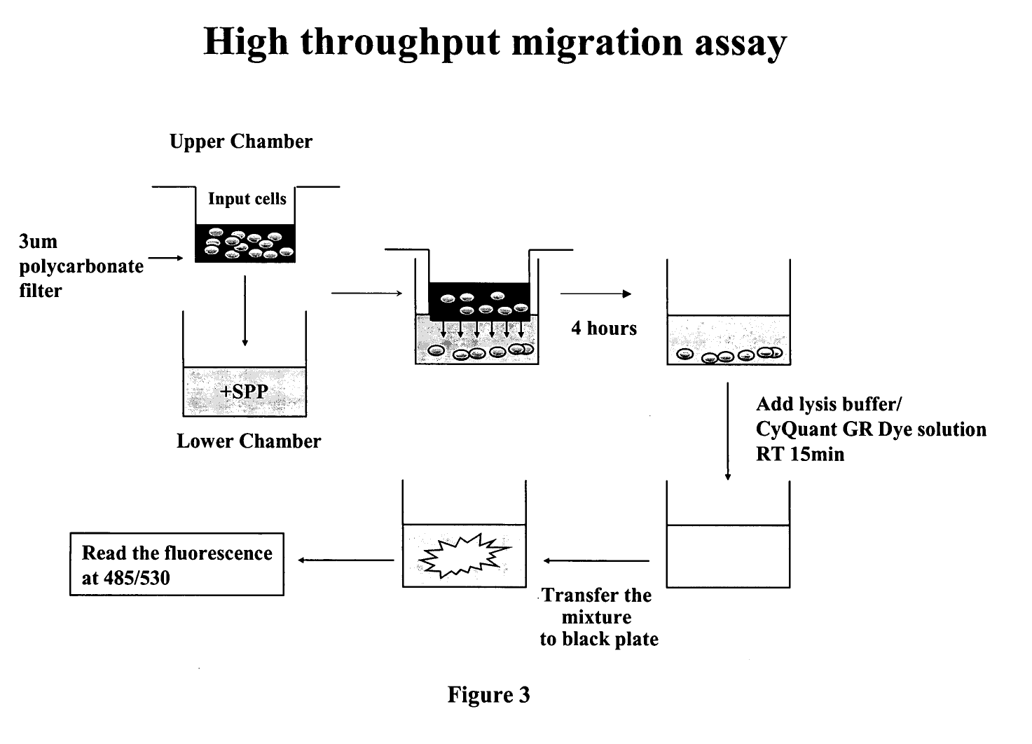

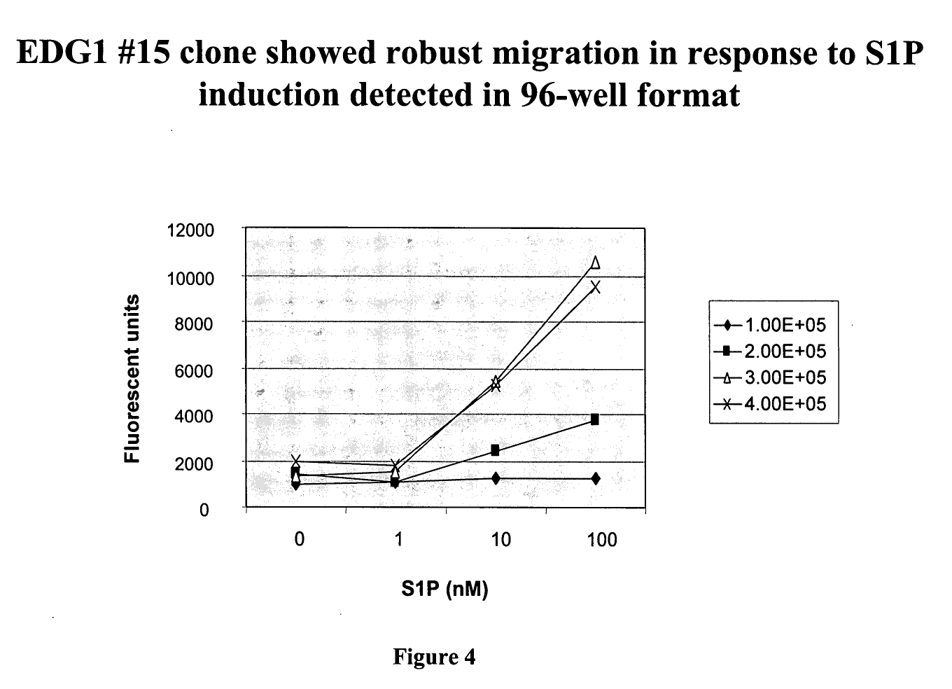

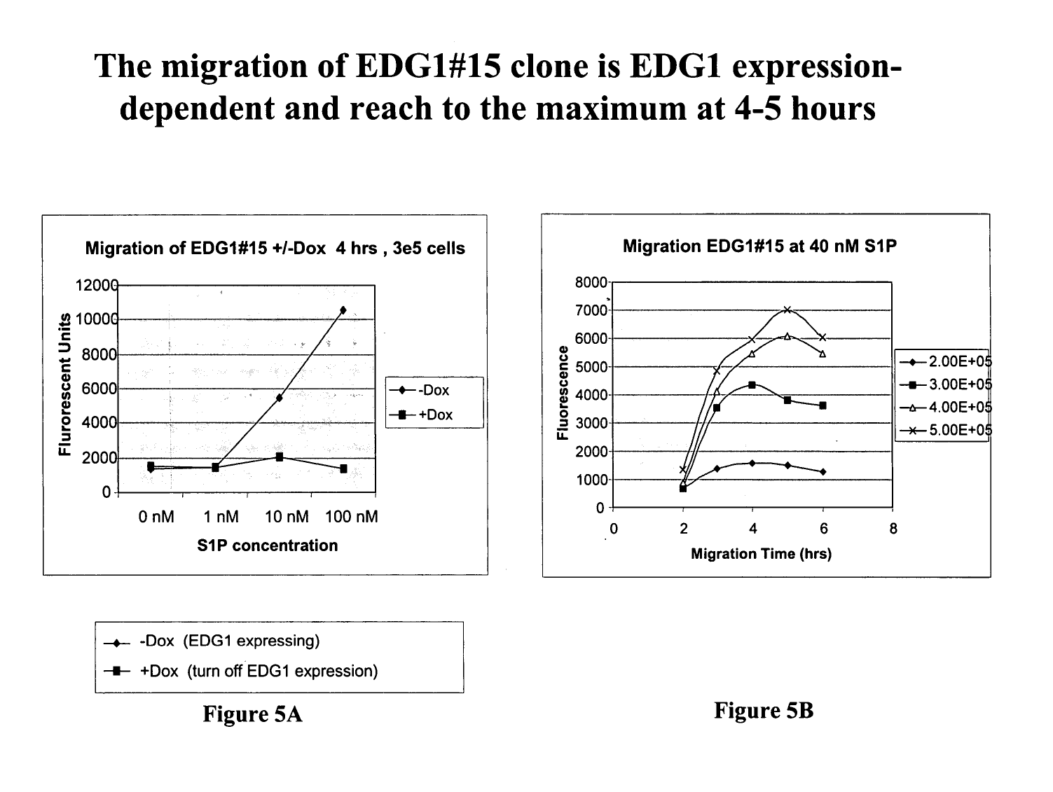

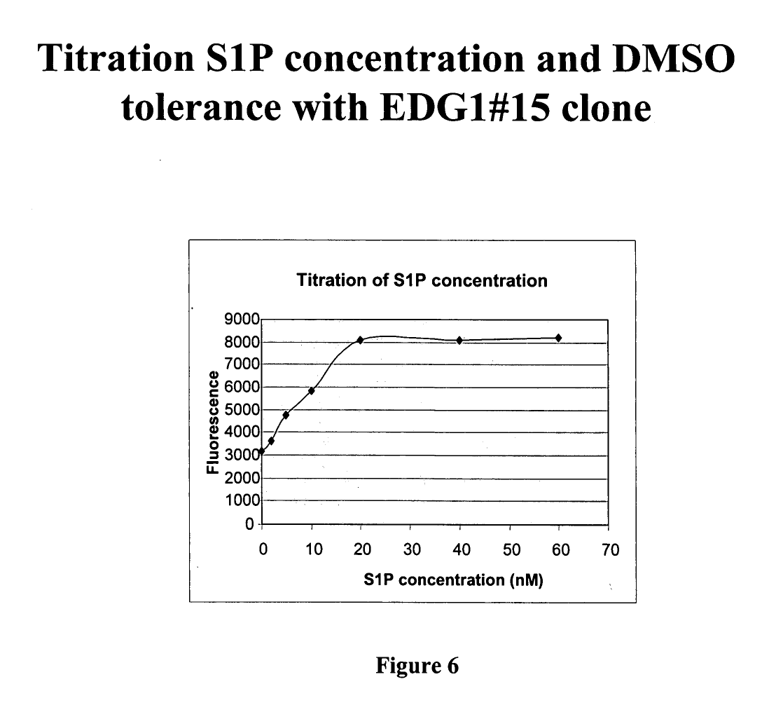

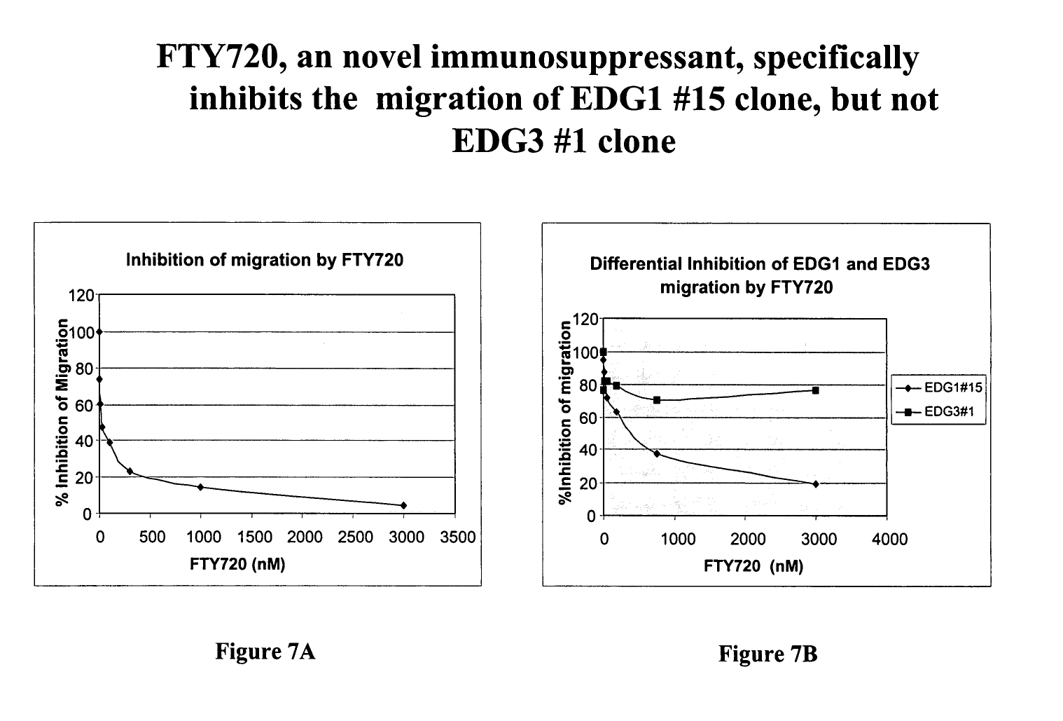

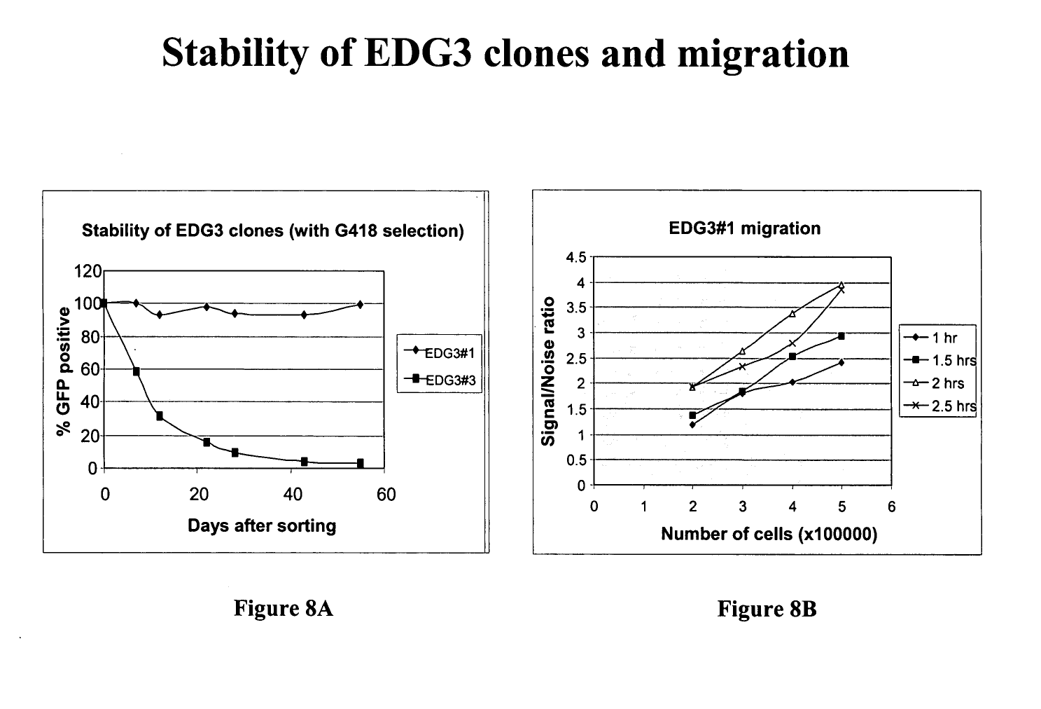

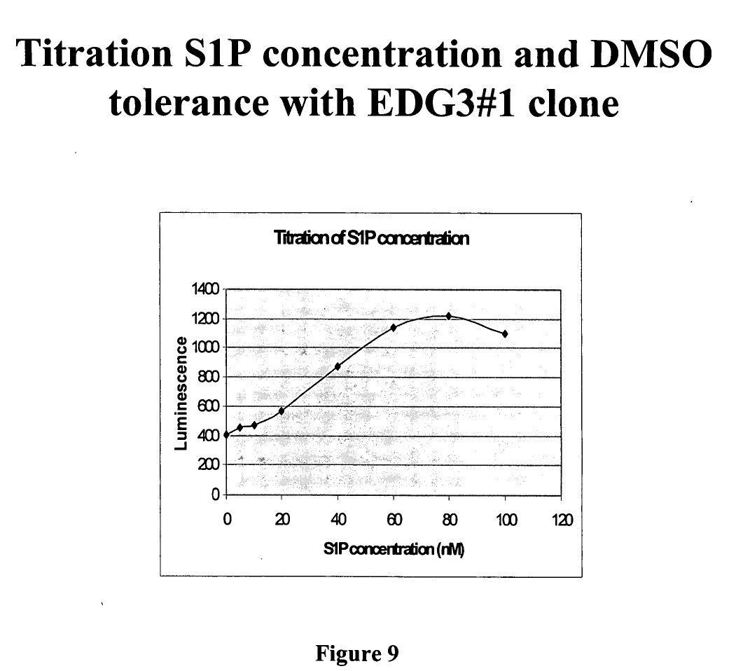

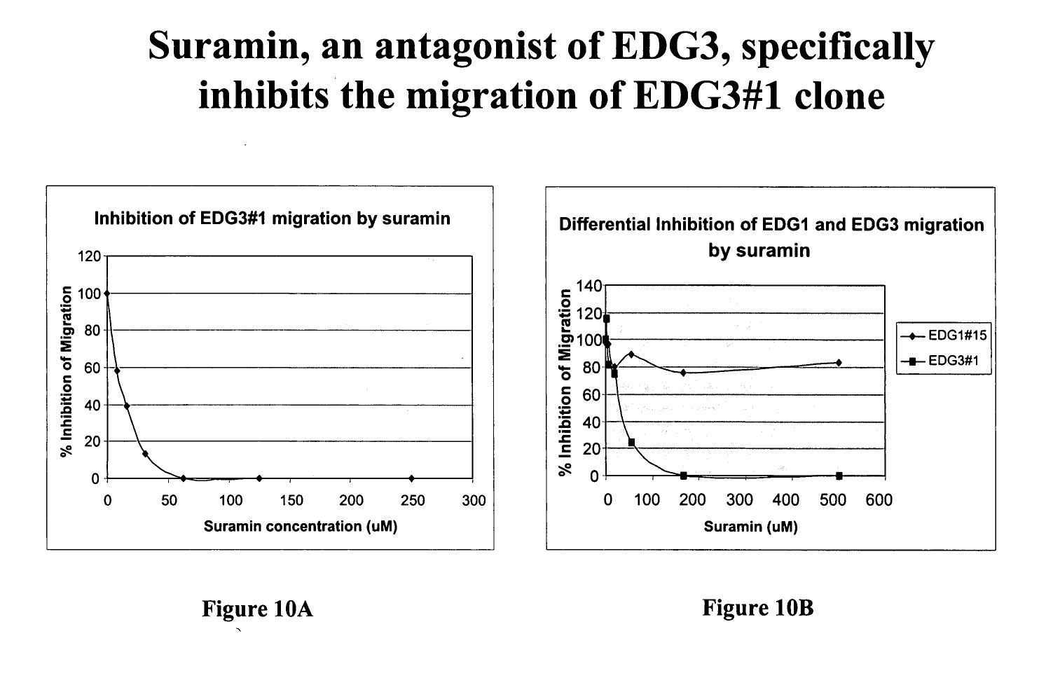

Exemplary wild type nucleic acid and protein sequences for additional members of the EDG family are provided by the following OMIM reference numbers (see also FIG. 2 for exemplary amino acid sequences of EDG family members):

For EDG-2, OMIM reference number 602282. The GenBank accession numbers for exemplary nucleotide and amino acids sequences are NM_{—}001401, XM_{—}005557, XM_{—}036690, XM_{—}036691, NP_{—}001392, XP_{—}5557, XP_{—}036690, XP_{—}036691, AAC00530, AAC51139, CAA70686, and CAA70687. (see, e.g., An et al., Molec. Pharm. 54:881-888 (1998); An et al., Biochem. Biophys. Res. Commun. 231:619-622 (1997); Contos et al., Genomics 51:364-378 (1998); Hecht et al., J. Cell. Biol. 135:1071-1083 (1996); and Moolenaar et al., Curr. Opin. Cell Biol. 9:168-173 (1997)).

For EDG-3, OMIM reference number 601965. The GenBank accession numbers for exemplary nucleotide and amino acids sequences are NM_{—}005226, NP_{—}005217, CAA58744 and AAC51906. (see, e.g., An et al., FEBS Lett. 417:279-282 (1997); and Yamaguchi et al., Biochem. Biophys. Res. Commun. 227:608-614 (1996)).

For EDG-4, OMIM reference number 605110. The GenBank accession numbers for exemplary nucleotide and amino acids sequences are NM_{—}004720, XM_{—}012893, XM_{—}048494, XM_{—}048495, NP_{—}004711, XP_{—}012893, XP_{—}048494, XP_{—}048495, AAB61528, AAC27728 and AAF43409. (see, e.g., An et al., J. Biol. Chem. 273:7906-7910 (1998); An et al., Molec. Pharm. 54:881-888 (1998); Contos et al., Genomics 64:155-169 (2000); and Goetzl et al., J. Immunol. 164:4996-4999 (2000)).

For EDG-5, OMIM reference number 605111. The GenBank accession numbers for exemplary nucleotide and amino acids sequences are NM_{—}004230, XM_{—}008898, NP_{—}004221, XP_{—}008898, and AAC98919. (see, e.g., An et al., J. Biol. Chem. 275:288-296 (2000); Kupperman et al., Nature 406:192-195 (2000); and MacLennan et al., Molec. Cell. Neurosci. 5:201-209 (1994)).

For EDG-6, OMIM reference number 603751. The GenBank accession numbers for exemplary nucleotide and amino acids sequences are NM_{—}003775, XM._{—}009219, NP_{—}003766, XP_{—}009219, and CAA04118. (see, e.g., Graler et al., Genomics 53:164-169 (1998); and Jedlicka et al., Cytogenet. Cell. Genet. 65:140 (1994)).

For EDG-7, OMIM reference number 605106. The GenBank accession numbers for exemplary nucleotide and amino acids sequences are NM_{—}012152, XM_{—}002057, XM_{—}035234, NP_{—}036284, XP._{—}002057, XP_{—}035234, AAD56311, AAF00530, and AAF91291. (see, e.g., Bandoh et al, J. Biol. Chem. 274:27776-27785 (1999)).

For EDG-8, OMIM reference number 605146. The GenBank accession numbers for exemplary nucleotide and amino acids sequences are NM._{—}030760, XM_{—}049584, NP_{—}110387, XP_{—}049584, and AAG3813. (see, e.g., Im et al., J. Biol. Chem. 275:14281-14286 (2000)).

As described above, EDG proteins have “G-protein coupled receptor activity,” e.g., they bind to G-proteins in response to extracellular stimuli, such as ligand binding, and promote production of second messengers such as IP3, cAMP, and Ca²⁺ via stimulation of enzymes such as phospholipase C and adenylate cyclase. Such activity can be measured in a heterologous cell, by coupling a GPCR (or a chimeric GPCR) to a G-protein, e.g., a promiscuous G-protein such as Gal 5, and an enzyme such as PLC, and measuring increases in intracellular calcium using (Offermans & Simon, J. Biol. Chem. 270:15175-15180 (1995)). Receptor activity can be effectively measured, e.g., by recording ligand-induced changes in [Ca²⁺]ᵢ and calcium influx using fluorescent Ca²⁺-indicator dyes and fluorometric imaging.

G protein coupled receptors are glycoproteins that share certain structural similarities (see, e.g., Gilman, Ann. Rev. Biochem. 56:615-649 (1987), Strader et al., The FASEB J. 3:1825-1832 (1989), Kobilka et al., Nature 329:75-79 (1985), and Young et al., Cell 45:711-719 (1986)). For example, G protein coupled receptors have an extracellular domain, seven hydrophobic stretches of about 20-25 amino acids in length interspersed with eight hydrophilic regions (collectively known as the transmembrane domain), and a cytoplasmic tail. Each of the seven hydrophobic regions forms a transmembrane alpha helix, with the intervening hydrophilic regions forming alternatively intracellular and extracellular loops. The third cytosolic loop between transmembrane domains five and six is involved in G-protein interaction. These transmembrane hydrophobic domains, hydrophilic loop domains, extracellular domains, and cytoplasmic tail domains can be structurally identified using methods known to those of skill in the art, such as sequence analysis programs that identify hydrophobic and hydrophilic domains (see, e.g., Kyte & Doolittle, J. Mol. Biol. 157:105-132 (1982)). Such domains are useful for making chimeric proteins and for in vitro assays of the invention (see, e.g., WO 94/05695 and U.S. Pat. No. 5,508,384). Such domains are also considered “fragments” of EDG proteins, and as such are useful in the assays of the invention, e.g., for ligand binding studies, or for signal transduction studies using chimeric proteins. Ligands for the EDG family are known in the art and include SPP, LPA, and GTP.

In another embodiment, migration molecules used in the methods of the invention are chemokine receptors. Chemokines are a large family of chemotactic cytokines that direct the trafficking and migration of leukocytes within the immune system. Chemokines mediate their activity through a large family of G-protein coupled receptors, the chemokine receptors. Chemokine receptors and chemokines are described in, for example, Cascieri and Springer (2000) Opinions in Chemical Biology 4:420-427, the contents of which are incorporated herein by reference. Chemokine receptors include CCR1 (GenBank Accession No.: GI:4502630), CCR2 (GenBank Accession No.: GI:15451896 or GI:4757937), CCR3 (GenBank Accession No.: GI:30581168 or GI:30581169), CCR4 (GenBank Accession No.: GI:39760188), CCR5 (GenBank Accession No.: GI:4502638), CCR6 (GenBank Accession No.: GI:37187859 or GI:37188164), CCR7 (GenBank Accession No.: GI:30795213), CCR8 (GenBank Accession No.: GI: 13929430), CCR9 (GenBank Accession No.: GI: 14043043 or GI: 14043041), CCR10 (GenBank Accession No.: GI:7546844), CCR11 (GenBank Accession No.: GI: 15919090), CXCR1 (GenBank Accession No.: GI:7209686), CXCR2 (GenBank Accession No.: GI:7209690, CXCR3 (GenBank Accession No.: GI:7209698), CXCR4 (GenBank Accession No.: GI:4503174), CXCR5 (GenBank Accession No.: GI: 14589868), CX3CR1 (GenBank Accession No.: GI:20380136), and XCR1 (GenBank Accession No.: GI:30526191).

Chemokine ligands are known in the art. Classically, the chemokine superfamily is defined by four conserved cysteines that form two disulfide bonds, and can be structurally subdivided into two major branches on the basis of the spacing of the first cysteine pair. Chemokines in which these residues are adjacent, such as RANTES and MIP-1alpha, form the CC subfamily, and those which are separated by a single amino acid, such as IL-8 and IP-10, comprise the CXC subfamily. Additional variants of these motifs exist. For example, there is at least one chemokine in which the cysteines are separated by three residues (CX2C), and one that lacks the first cysteine in the pair (C). Some chemokine receptors (GPCRs) are specific and bind with a single chemokine, whereas others, the so-called shared receptors, bind multiple ligands within, but not between, the CC or CXC branches. The chemokine family includes the following: MIP-1α (GenBank Accession No.: GI:3252190), RANTES (GenBank Accession No.: GI:339420), MCP-2 (GenBank Accession No.: GI:1905800), MCP-3 (GenBank Accession No.:GI:3928270), MCP4 (GenBank Accession No.:GI:2689216), MDC (GenBank Accession No.: GI:1931580), TARC (GenBank Accession No.: GI:5102777), eotaxin (GenBank Accession No.: GI:1552240), eotaxin-2 (GenBank Accession No.: GI:22165426), eotaxin-3 (GenBank Accession No.:GI:5921130), MIP-3α (GenBank Accession No.: GI:23345788), MIP-3β(GenBank Accession No.: GI:1791002), MIP-5 (GenBank Accession No.: GI:34335181), MPIF-1 (GenBank Accession No.: GI:22538805), HCC-1 (GenBank Accession No.: GI:34335177), lymphotaxin (GenBank Accession No.: GI:4938297), fractalkine (GenBank Accession No.: GI:19745169), ILB, GCP2 (GenBank Accession No.: GI:4506850), Groα (GenBank Accession No.: GI:4504152), Groβ (GenBank Accession No.: GI:4504154), ENA78, NAP-2 (GenBank Accession No.: GI:129874), IP10 (GenBank Accession No.:GI:4504700), Mig (GenBank Accession No.: GI:6678879), ITAC (GenBank Accession No.: GI: 14790145), SDF-1 (GenBank Accession No.: GI:40316922), BLC (GenBank Accession No.: GI:2911375), ELC (GenBank Accession No.: GI:2189952), SLC (GenBank Accession No.: GI:22165425), CTACK (GenBank Accession No.: GI:22165428), TECK (GenBank Accession No.: GI:22538795), I-308, TARK, and MDC (GenBank Accession No.: GI:22538803).

Migration molecules, as described herein, also include adhesion molecules, e.g., selectins, integrins, cadherins, and certain members of the immunoglobulin superfamily of molecules. Selectins are multifunctional adhesion molecules that mediate the initial interactions between circulating leukocytes and cells of the endothelium that is manifested as leukocyte rolling. Selectins are involved in normal lymphocyte homing, leukocyte recruitment during inflammatory responses, carbohydrate ligand biosynthesis and adhesion-mediated signaling. In addition, selectins have been identified as targets for drug delivery in the development of new anti-inflammatory therapeutics, anti-atherosclerosis therapeutics, and anti-cancer therapy. Selectins are described in, for example, Ehrhardt C. (2004) Adv Drug Deliv Rev. Mar 3;56(4):52749, the contents of which are incorporated herein by reference. Selectin molecules include, P-selectin (GenBank Accession No.: GI:6031196), E-selectin (GenBank Accession No.: GI:4506870), and L-selectin (GenBank Accession No.: GI:5713320).

Ligands of selectins are known in the art and generally comprise at least in part of a carbohydrate moiety. P-selectin binds to carbohydrates containing the non-sialated form of the Lewis^{x} blood group antigen and with higher affinity to sialyl Lewis^{x}, which is contained within PSGL-1, a known ligand of P-selectin. E-selectin also binds PSGL-1. L-selectin on lymphocytes binds to sulphated ligands expressed by the specialized endothelial cells of high endothelial venules (HEVs). Selectin ligands are described in, for example, McEver (2004) Ernst Schering Res Found Workshop. (44): 13747 and Kannagi R (2002) Curr Opin Struct Biol. Oct;12(5):599-608 and van Zante A, Rosen S D. (2003) Biochem Soc Trans. Apr;31(2):313-7.

Integrins are cell surface membrane glycoproteins which function as adhesion receptors in cell-extracellular matrix interactions. Integrins play a role in the regulation of various processes including proliferation, differentiation, and cell migration. Integrins are described in, for example, Pozzi A. (2003) Nephron Exp Nephrol. 94(3):e77-84, the contents of which are incorporated herein by reference. Integrins include α1β1 (α1, GenBank Accession No.:GI:31657141, β1, GI:19743822), α2β1 (α2, GenBank Accession No.:6006008), α3β1 (α3, GenBank Accession No.:6006010), α4β1 (α4, GenBank Accession No.:6006032), α5 β1 (α5, GenBank Accession No.:4504750), α6 β1 (α6, GenBank Accession No.:5726562), α7β1 (α7, GenBank Accession No.:GI:4504752), α8β1 (VLA-8) (α8, GenBank Accession No.: GI:37551030), α9β1 (α9, GenBank Accession No.: GI: 11321594), αVβ3 (αV, GenBank Accession No.: GI:9944821; β3, GenBank Accession No.: GI:186502), αVβ1, αLβ2 (αL, GenBank Accession No.: GI:4504756), αMβ2 (αM, GenBank Accession No.: GI:6006013), αXβ2 (αX, GenBank Accession No.: GI:34452172), αIIβ3 (αII, GenBank Accession No.: GI:6006009), α6β3 (α6, GenBank Accession No.: GI:4557674; β3, GenBank Accession No.: GI:47078291), α6β4 (β4, GenBank Accession No.: GI:21361206), αVβ5 (β5, GenBank Accession No.: GI:34147573), αVβ6 (β6, GenBank Accession No.: GI:9966771), αVβ8 (β8, GenBank Accession No.: GI:4504778), α4β7 (β7, GenBank Accession No.: GI:4504776), αIELβ7 (αIEL, GenBank Accession No.: GI:6007850), and α11(GenBank Accession No.:GI:19923396).

Integrins can adhere an array of ligands. Common ligands are for example fibronectin and laminin, which are both part of the extracellular matrix or basal lamina's. Both of these ligands are recognized by multiple integrins. For adhesion to ligands, both integrin subunits are needed, as is the presence of cations. The alpha chain contains cation binding sites.

Osteopontin binds to cells via integrin and non-integrin receptors, and is a ligand for αvβ3, αvβ1, and αvβ5 integrins. Osteopontin supports the migration and adhesion of osteoclasts and osteoblasts and appears to be chemotactic to osteoprogenitor cells. Osteopontin is also elevated in sera from patients with advanced metastatic cancer and cellular transformation may lead to enhanced osteopontin expression and increased metastatic activity. Expression of antisense RNA in metastatic Ras transformed fibroblasts resulted in the reduction of the metastatic potential of these cells. The presence of a Gly-Arg-Gly-Asp-Ser (GRGDS) cell-surface receptor binding motif within the sequence of osteopontin suggests that osteopontin may be involved in cell attachment and spreading (Oldberg et al. (1986) Proc. Natl. Acad. Sci. USA 83:88 19; Oldberg et al. (1986) J. Biol. Chem. 263:19433-19436).

Integrins are also involved in the modulation of angiogenesis. It has been shown that ligation of α5β1 by fibronectin suppresses protein kinase A activation and permits the association of αvβ3 with the actin cytoskeleton as well as cellular migration. Integrin αvβ3, in contrast, is a promiscuous integrin with the potential to mediate migration on a host of extracellular matrix proteins with arginine-glycine-aspartic acid moieties, such as vitronectin, fibrinogen, collagen, von Willebrand's factor, and others (Kim, et al. (2000) J. Biol. Chem., Vol. 275, Issue 43, 33920-33928). Additional integrin ligands are known in the art and can be used in the methods of the invention.

Cadherins are calcium dependent cell adhesion proteins which are composed of a single protein chain that folds into a series of domains. They preferentially interact with themselves in a homophilic manner in connecting cells; cadherins may thus contribute to the sorting of heterogeneous cell types. For example, cadherins appear to be critical in segregating embryonic cells into tissues. Cadherins ultimately anchor cells through cytoplasmic actin and intermediate filaments. Cadherins include, for example, Cadherin E (1) (GenBank Accession No.:GI:14589887), Cadherin N (2) (GenBank Accession No.: GI: 14589888), Cadherin BR (12) (GenBank Accession No.: GI: 16445392), Cadherin P (3) (GenBank Accession No.: GI:45269142), Cadherin R (4) (GenBank Accession No:GI:14589892), Cadherin M (15) (GenBank Accession No.: GI:16507957), Cadherin VE (5) (CD144) (GenBank Accession No.: GI: 14589894), Cadherin T & H (13) (GenBank Accession No.: GI: 16507956), Cadherin OB (11) (GenBank Accession No.: GI: 16306531), Cadherin K (6) (GenBank Accession No.: GI: 15011911), Cadherin 7 (GenBank Accession No.: GI:16306488), Cadherin 8 (GenBank Accession No.: GI:16306538), Cadherin KSP (16) (GenBank Accession No.: GI: 16507958), Cadherin LI (17) (GenBank Accession No.: GI: 854174), Cadherin 18 (GenBank Accession No.: GI:16445394), Fibroblast 1 (19) (GenBank Accession No.: GI: 16933556), Cadherin Fibroblast 2 (20) (GenBank Accession No.: GI: 14270497), Cadherin Fibroblast 3 (21) (GenBank Accession No.: GI:14196452), Cadherin 23 (GenBank Accession No.:GI:18077850), Desmocollin 1 (GenBank Accession No.: G: 13435362), Desmocollin 2 (GenBank Accession No.:GI:40806176), Desmoglein 1 (GenBank Accession No.:GI:4503400), Desmoglein 2 (GenBank Accession No.:GI:4503402), Desmoglein 3 (GenBank Accession No.:GI:13435368), and Protocadherin 1, 2, 3, 7, 8, and 9 (GenBank Accession No.:GI:30411048, GI:6631101, GI:45243537).

The failure of cadherin is one of the key steps in the creation of metastases. In order to metastasize, tumor cells must gain the ability to separate from their neighbors and travel through the blood to distant sites. Cadherin function is lost in different ways in different cancers. Some have mutations that reduce the production of cadherin, stopping its function at the source. Other tumors have a mutation in the protein itself, destroying its adhesive function. Others create a protein-cutting enzyme that attacks cadherin. Whatever the mechanism, the integrity of the tissue is destroyed and free cancer cells are released, ready to invade healthy tissues. Cadherins are described in Goodsell D S (2002) Stem Cells 20:583-584, incorporated herein by reference.

Members of the immunoglobulin superfamily contain immunoglobulin-like domains and some are responsible for strong attachment and transendothelial migration of leukocytes, e.g., during inflammation. Certain immunoglobulin superfamily molecules are also involved in myelination and neurite outgrowth. Immunoglobulin superfamily molecules include, for example, Inter-Cellular Adhesion Molecule-1 (I-CAM-1) (CD54) (GenBank Accession No.:GI:4557877), Inter-Cellular Adhesion Molecule-2 (I-CAM-2) (CD102) (GenBank Accession No.: GI:13111858), Inter-Cellular Adhesion Molecule-3 (I-CAM-3) (CD50) (GenBank Accession No.: GI:12545399), and Vascular-Cell Adhesion Molecule (V-CAM) (GenBank Accession No.: GI:18201908), ALCAM (CD166) (GenBank Accession No.: GI:4502028), Basigin (CD147) (GenBank Accession No.: GI:31076332), BL-CAM (CD22) (GenBank Accession No.: GI:4502650), CD44 (GenBank Accession No.: GI: 180129), Lymphocyte function antigen-2 (LFA-2) (CD2) (GenBank Accession No.: GI:180093), LFA-3 (CD 58) (GenBank Accession No.: GI:466540), Major histocompatibility complex (MHC) molecules, MAdCAM-1 (GenBank Accession No.: GI:18780284), and platelet endothelial cell adhesion molecule-1 (PECAM) (CD31) (GenBank Accession No.: GI:598195). Members of the immunoglobulin superfamily are described in Barclay A N (2003) Semin Immunol. 15(4):215-23; Radi Z A, et al. (2001) J Vet Intern Med. 5(6):516-29; Huang Z, Li S, Komgold R (1997) Biopolymers 43(5):367-82; and Cotran R S, Mayadas-Norton (1998) T. Pathol Biol (Paris) 46(3): 164-70, the contents of which are incorporated herein by reference.

It is understood that the nucleotide and amino acid sequences of the molecules described herein are not limited to any particular exemplary GenBank Accession Number set forth herein.

A migration molecule, e.g., EDG, e.g., EDG1 and EDG3, selectin, integrin, or chemokine receptor molecule, is typically from a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules. The polypeptide further has the ability to bind its naturally occurring ligand, e.g., SPP or LPA, as well as other naturally occurring and synthetic ligands and their analogs, including sphingolipid-like compounds.

The terms “migration molecule” “migration protein” or a fragment thereof, or a nucleic acid encoding a “migration molecule” or “migration protein” or a fragment thereof refer to nucleic acid and polypeptide polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to an amino acid sequence encoded by a migration molecule; (2) specifically bind to antibodies, e.g., polyclonal antibodies, raised against an immunogen comprising an amino acid sequence encoded by a migration molecule, immunogenic fragments thereof, and conservatively modified variants thereof; (3) specifically hybridize under stringent hybridization conditions to an anti-sense strand corresponding to a nucleic acid sequence encoding a migration protein, or their complements, and conservatively modified variants thereof; (4) have a nucleic acid sequence that has greater than about 60% sequence identity, 65%, 70%, 75%, 80%, 85%, 90%, preferably 91%, 92%, 93%, 94%, 95%, 96%, 97%, 98% or 99%, or higher nucleotide sequence identity, preferably over a region of at least about 25, 50, 100, 200, 500, 1000, or more nucleotides, to a nucleotide sequence of a migration molecule or their complements. The migration molecules of the invention further have the ability to bind their naturally occurring ligand, e.g., SPP, and synthetic ligands and their analogs, including, for example, sphingolipid-like compounds.

B. Reporter Genes

In an especially preferred embodiment, a reporter gene used in the methods of the invention encodes a detectable protein that can be used as a direct label, for example a detection gene for sorting the cells or for cell enrichment by FACS. In this embodiment, the protein product of the reporter gene itself can serve to distinguish cells that are expressing the reporter gene. In this embodiment, suitable reporter genes include those encoding a luciferase gene from firefly, Renilla, or Ptiolosarcus, as well as genes encoding green fluorescent protein (GFP; Chalfie, M. et al. (1994) Science 263: 802-05; and EGFP; Clontech—Genbank Accession Number U55762), blue fluorescent protein (BFP; Quantum Biotechnologies, Inc. 1801 de Maisonneuve Blvd. West, 8th Floor, Montreal (Quebec) Canada H3H 1J9; Stauber, R. H. (1998) Biotechniques 24: 462-71; Heim, R. et al. (1996) Curr. Biol. 6: 178-82), enhanced yellow fluorescent protein (EYFP; 1. Clontech Laboratories, Inc., 1020 East Meadow Circle, Palo Alto, Calif. 94303), luciferase (Kennedy, H. J. et al. (1999) J. Biol. Chem. 274: 13281-91), Renilla reniformis GFP (WO 99/49019), Ptilosarcus gumeyi GFP (WO 99/49019; U.S. Ser. No. 60/164,592; U.S. Ser. No. 09/710,058; U.S. Ser. No. 60/290,287), Renilla mulleris GFP (WO 99/49019; U.S. Ser. No. 60/164,592; U.S. Ser. No. 09/710,058; U.S. Ser. No. 60/290,287); GFP homologue from Anthozoa species (Nat. Biotech., 17:969-973, 1999); β-galactosidase (Nolan, G. et al. (1988) Proc. Natl. Acad. Sci. USA 85: 2603-07), β-glucouronidase (Jefferson, R. A. et al. (1987) EMBO J. 6: 3901-07; Gallager, S., “GUS Protocols: Using the GUS Gene as a reporter of gene expression,” Academic Press, Inc., 1992), and secreted form of human placental alkaline phosphatase, SEAP (Cullen, B. R. et al. (1992) Methods Enzymol. 216: 362-68). In a preferred embodiment, the codons of the reporter genes are optimized for expression within a particular organism, especially mammals, and particularly preferred for humans (see Zolotukhin, S. et al. (1996) J. Virol. 70: 4646-54; U.S. Pat. No. 5,968,750; U.S. Pat. No. 6,020,192; U.S. S. No. 60/290,287, all of which are expressly incorporate by reference).

The green fluorescent protein from Aequorea Victoria is a 238 amino acid protein. The crystal structure of the protein and of several point mutants has been solved (Ormo et al., Science 273, 1392-5,1996; Yang et al., Nature Biotechnol. 14, 1246-51, 1996). The fluorophore, consisting of a modified tripeptide, is buried inside a relatively rigid β-can structure, where it is almost completely protected from solvent access. The fluorescence of this protein is sensitive to a number of point mutations (Phillips, G. N., Curr. Opin. Struct. Biol. 7, 821-27, 1997). The fluorescence appears to be a sensitive indication of the preservation of the native structure of the protein, since any disruption of the structure allowing solvent access to the fluorophoric tripeptide will quench the fluorescence.

The Renilla GFP used in the present invention preferably has significant homology to the wild-type Renilla GFP protein as depicted in WO 99/49019, hereby incorporated by reference in its entirety.

Alternatively, the reporter gene encodes a protein that will bind a label that can be used as the basis of the cell enrichment (sorting); that is, the reporter gene serves as an indirect label or detection gene. In this embodiment, the reporter gene should encode a cell-surface protein. For example, the reporter gene may be any cell-surface protein not normally expressed on the surface of the cell, such that secondary binding agents serve to distinguish cells that contain the reporter gene from those that do not. Alternatively, albeit non-preferably, reporters comprising normally expressed cell-surface proteins could be used, and differences between cells containing the reporter construct and those without could be determined. Thus, secondary binding agents bind to the reporter protein. These secondary binding agents are preferably labeled, for example with fluorophores, and can be antibodies, haptens, etc. For example, fluorescently labeled antibodies to the reporter gene can be used as the label. Similarly, membrane-tethered streptavidin could serve as a reporter gene, and fluorescently-labeled biotin could be used as the label, i.e. the secondary binding agent. Alternatively, the secondary binding agents need not be labeled as long as the secondary binding agent can be used to distinguish the cells containing the construct; for example, the secondary binding agents may be used in a column, and the cells passed through, such that the expression of the reporter gene results in the cell being bound to the column, and a lack of the reporter gene (i.e. inhibition), results in the cells not being retained on the column. Other suitable reporter proteins/secondary labels include, but are not limited to, antigens and antibodies, enzymes and substrates (or inhibitors), etc.

In one embodiment, the reporter gene is a survival gene that serves to provide a nucleic acid (or encode a protein) without which the cell cannot survive, such as drug resistance genes. In this embodiment, expressing the survival gene allows selection of cells by identifying cells that survive, for example in presence of a selection drug. Examples of drug resistance genes include, but are not limited to, puromycin resistance (puromycin-N-acetyl-4ransferase) (de la Luna, S. and Ortin, J. Methods Enzymol.(1992) 216:376-385), G418 neomycin resistance gene, hygromycin resistance gene (hph), and blasticidine resistance genes (bsr, brs, and BSD) (Pere-Gonzalez, et al., Gene (1990).86: 129-134; Izumi et al., Exp. Cell Res. (1991) 197: 229-233; Itaya et al. (1990) J. Biochem. 107: 799-801; Kimura, et al. Mol. Gen. Genet. (1994) 242:121-129). In addition, generally applicable survival genes are the family of ATP-binding cassette transporters, including multiple drug resistance gene (MDR1) (see Kane et. al. (1988) Mol. Cell. Biol. 8: 3316 and Choi et al. (1988) Cell 53: 519), multidrug resistance associated proteins (MRP) (Bera T. K. et al. (2001) Mol. Med. 7:509-16), and breast cancer associated protein (BCRP or MXR) (Tan B. et al. (2000) Curr. Opin. Oncol. 12:450-8). When expressed in cells, these selectable genes can confer resistance to a variety of anti-cancer drugs (i.e. methotrexate, colchicine, tamoxifen, mitoxanthrone, and doxorubicin). The choice of reporter gene will depend on, for example, the cell type used.

In one embodiment, the reporter gene is a cell cycle gene, that is, a gene that causes alterations in the cell cycle. For example, Cdk interacting protein p21 (see Harper et al. (1993) Cell 75: 805-816), which inhibits cyclin dependent kinases, does not cause cell death but causes cell-cycle arrest. Thus, expressing the p21 allows selection for regulators of promoter activity or regulators of p21 activity based on detecting cells that grow out much more quickly due to low p21 activity, either through inhibiting promoter activity or inactivation of p21 protein activity. As will be appreciated by those in the art, it is also possible to configure the system to select cells based on their inability to grow out due to increased p21 activity.

In yet another preferred embodiment, the reporter gene encodes a cellular biosensor. By a cellular biosensor herein is meant a gene product that when expressed within a cell can provide information about a particular cellular state. Biosensor proteins allow rapid determination of changing cellular conditions, for example Ca⁺² levels in the cell, pH within cellular organelles, and membrane potentials (see Miesenbock, G. et al. (1998) Nature 394: 192-95). An example of an intracellular biosensor is Aequorin, which emits light upon binding to Ca⁺² ions. The intensity of light emitted depends on the Ca⁺² concentration, thus allowing measurement of transient calcium concentrations within the cell. When directed to particular cellular organelles by fusion partners, as more fully described below, the light emitted by Aequorin provides information about Ca⁺² concentrations within the particular organelle. Other intracellular biosensors are chimeric GFP molecules engineered for fluorescence resonance energy transfer (FRET) upon binding of an analyte, such as Ca⁺² (Miyawaki, A. et al. (1997) Nature 388: 882-87; Miyakawa, A. et al. (1997) Mol. Cell. Biol. 8: 2659-76). For example, Camelot consists of blue or cyan mutant of GFP, calmodulin, CaM binding domain of myosin light chain kinase, and a green or yellow GFP. Upon binding of Ca⁺² by the CaM domain, FRET occurs between the two GFPs because of a structural change in the chimera. Thus, FRET intensity is dependent on the Ca+2 levels within the cell or organelle (Kerr, R. et al. Neuron (2000) 26: 583-94). Other examples of intracellular biosensors include sensors for detecting changes in cell membrane potential (Siegel, M. et al. (1997) Neuron 19: 735-41; Sakai, R. (2001) Eur. J. Neurosci. 13: 2314-18), monitoring exocytosis (Miesenbrock, G. et al. (1997) Proc. Natl. Acad. Sci. USA 94: 3402-07), and measuring intracellular/organellar ATP concentrations via luciferase protein (Kennedy, H. J. et al. (1999) J. Biol. Chem. 274: 13281-91). These biosensors find use in monitoring the effects of various cellular effectors, for example pharmacological agents that modulate ion channel activity, neurotransmitter release, ion fluxes within the cell, and changes in ATP metabolism.

Other intracellular biosensors comprise detectable gene products with sequences that are responsive to changes in intracellular signals. These sequences include peptide sequences acting as substrates for protein kinases, peptides with binding regions for second messengers, and protein interaction sequences sensitive to intracellular signaling events (see for example, U.S. Pat. No. 5,958,713 and U.S. Pat. No. 5,925,558). For example, a fusion protein construct comprising a GFP and a protein kinase recognition site allows measuring intracellular protein kinase activity by measuring changes in GFP fluorescence arising from phosphorylation of the fusion construct. Alternatively, the GFP is fused to a protein interaction domain whose interaction with cellular components are altered by cellular signaling events. For example, it is well known that inositol-triphosphate (InsP3) induces release of Ca+2 from intracellular stores into the cytoplasm, which results in activation of a kinases responsible for regulating various cellular responses. The precursor to InsP3 is phosphatidyl-inositol 4,5-bisphosphat (PtdInsP₂), which is localized in the plasma membrane and cleaved by phospholipase C (PLC) following activation of an appropriate receptor. Many signaling enzymes are sequestered in the plasma membrane through pleckstrin homology domains that bind specifically to PtdInsP₂. Following cleavage of PtdInsP₂, the signaling proteins translocate from the plasma membrane into the cytosol where they activate various cellular pathways. Thus, a reporter molecule such as GFP fused to a pleckstrin domain will act as a intracellular sensor for phospholipase C activation (see Haugh, J. M. et al. (2000) J. Cell. Biol. 15: 1269-80; Jacobs, A. R. et al. (2001) J. Biol. Chem. 276: 40795-802; and Wang, D. S. et al. (1996) Biochem. Biophys. Res. Commun. 225: 420-26). Other similar constructs are useful for monitoring activation of other signaling cascades and applicable as assays in screens for candidate agents that inhibit or activate particular signaling pathways.

In one embodiment of the invention, a vector may comprise more than one selection gene, e.g., a first and a second selection gene. In certain embodiment, it may be desirable to fuse the first and second selection gene such that transcription from a promoter operably linked to the first selection gene results in a single transcript encoding the first and second selection genes and further comprising a site which allows for functional separation of the two selection genes. Such functional separation can be achieved, e.g., by the use of internal ribosome entry sites (IRES) or proteolytic cleavage sites, e.g., 2a sites.

When the retroviral vectors express fusion nucleic acids encoding a plurality of genes of interest, e.g., a test agent and a migration molecule, the separation sequence may be operably linked to the first gene of interest and second gene of interest such that the fusion nucleic acid is capable of producing separate protein products of interest. Thus, in a preferred embodiment, the separation sequence is placed in between the first gene of interest and the second gene of interest. As will be appreciated by those skilled in the art, use of separation sequences based on protease recognition sites or Type 2A sequences requires that the fusion nucleic acid comprising the first gene of interest, separation sequence, and second gene of interest to be in-frame. By “in-frame” herein is meant that the fusion nucleic acid encodes a continuous single polypeptide comprising the protein encoded by the first gene of interest, protein encoded by the separation sequence, and protein encoded by the second gene of interest. Standard recombinant DNA techniques may be used for placing the components of the fusion nucleic to encode a contiguous single polypeptide. Peptide linkers may be added to the separation sequence to facilitate the separation reaction or limit structural interference of the separation sequence on the gene of interest (and vice versa). Preferred linkers are (Gly)ₙ linkers, where n is 1 or more, with n being two, three, four, five or six, although linkers of 7-10 or amino acids are also possible.

As is appreciated by those in the art, use of IRES type sequences does not require the first gene of interest, separation sequence, and second gene of interest to be in frame since IRES elements function as internal translation initiation sites. Accordingly, fusion nucleic acids using IRES elements have the genes of interest arranged in a cistronic structure. That is, transcription of the fusion nucleic acid produces a cistronic mRNA that encodes both first gene of interest and second gene of interest with the IRES element controlling translation initiation of the downstream gene of interest. Alternatively, separate IRES sequences may control the upstream and downstream gene of interest.

The subject vectors may also comprise enhancers of IRES mediated translation initiation. IRES initiated translation may be enhanced by any number of methods. Cellular expression of virally encoded proteases that cleaves eIF4F to remove CAP-binding activity from the 40S ribosome complexes may be employed to increase preference for IRES translation initiation events. These proteases are found in some Picornaviruses and can be expressed in a cell by introducing the viral protease gene by transfection or retroviral delivery (Roberts, L. O. (1998) RNA 4: 520-29). Other enhancers adaptable for use with IRES elements include cis-acting elements, such as 3′ untranslated region of hepatitis C virus (Ito, T. et al. (1998) J. Virol. 72: 8789-96) and polyA segments (Bergamini, G. et al. (2000) RNA 6: 1781-90), which may be included as part of the fusion nucleic acid of the present invention. In addition, preferential use of cellular IRES sequences may occur when CAP dependent mechanisms are impaired, for example by dephosphorylation of 4E-BP, proteolytic cleavage of eIF4G, or when cells are placed under stress by .gamma.-irradiation, amino acid starvation, or hypoxia. Thus, in addition to the methods described above, IRES enhancing procedures include activation or introduction of 4E-BP targeted phosphatases or proteases of eIF4G. Alternatively, the cells are subjected to stress conditions described above. Other trans-acting IRES enhancers include heterogeneous nuclear ribonucleoprotein (hnRNP, Kaminski, A. et al. (1998) RNA 4: 626-38), PTB hnRNP E2/PCBP2 (Walter, B. L. et al. (1999) RNA 5: 1570-85), La autoantigen (Meerovitch, K. et al. (1993) J. Virol. 67: 3798-07), unr (Hunt, S. L. et al. (1999) Genes Dev. 13: 43748), ITAF45/Mpp1 (Pilipenko, E. V. et al. (2000) Genes Dev. 14: 2028-45), DAP5/NAT1/p97 (Henis-Korenblit, S. et al. (2000) Mol. Cell. Biol. 20: 496-506), and nucleolin (Izumi, R. E. et al. (2001) Virus Res. 76: 17-29).

These factors may be introduced into a cell either alone or in combination. Accordingly, various combinations of IRES elements and enhancing factors are used to effect a separation reaction. In another preferred embodiment, the separation sites are Type 2A separation sequences. By “Type 2A” sequences herein is meant nucleic acid sequences that when translated inhibit formation of peptide linkages during the translation process. Type 2A sequences are distinguished from IRES sequences in that 2A sequences do not involve CAP independent translation initiation. Without being bound by theory, Type 2A sequences appear to act by disrupting peptide bond formation between the nascent polypeptide chain and the incoming activated tRNA^{PRO} (Donnelly, M. L. et al. (2001) J. Gen. Virol 82: 1013-25). Although the peptide bond fails to form, the ribosome continues to translate the remainder of the RNA to produce separate peptides unlinked at the carboxy terminus of the 2A peptide region. An advantage of Type 2A separation sequences is that near stoichiometric amounts of first protein of interest and second protein of interest are made as compared to IRES elements. Moreover, Type 2A sequences do not appear to require additional factors, such as proteases that are required to effect separation when using protease recognition sites. Although the exact mechanism by which Type 2A sequences function is unclear, practice of the present invention is not limited by the theorized mechanisms of 2A separation sequences. Preferred Type 2A separation sequences are those found in cardioviral and apthoviral genomes, which are approximately 21 amino acids long and have the general sequence XXXXXXXXXXLXXXDXEXNPG (SEQ ID NO:1), where X is any amino acid. Disruption of peptide bond formation occurs between the underlined carboxy terminal glycine (G) and proline (P). These 2A sequences are found, among others, in the apthovirus Foot and Mouth Disease Virus (FMDV), cardiovirus Theiler's murine encephalomyelitis virus (TME), and encephalomyocarditis virus (EMC). Various viral Type 2A sequences are known in the art. The 2A sequences function in a wide range of eukaryotic expression systems, thus allowing their use in a variety of cells and organisms. Accordingly, inserting these 2A separation sequences in between the nucleic acids encoding the first gene of interest and second gene of interest, as more fully explained below, will lead to expression of separate protein products of the first gene of interest and the second gene of interest.

In another embodiment, the present invention contemplates mutated versions or variants of Type 2A sequences. By “mutated” or “variant” or grammatical equivalents herein is meant deletions, insertions, transitions, transversions of nucleic acid sequences that exhibit the same qualitative separating activity as displayed by the naturally occurring analogue, although preferred mutants or variants have higher efficient separating activity and efficient translation of the downstream gene of interest. Mutant variants include changes in nucleic acid sequence that do not change the corresponding 2A amino acid sequence, but incorporate frequently used codons (i.e., codon optimized) to allow efficient translation of the 2A region (see Zolotukin, S. et al. (1996) J. Virol. 70: 4646-54). In another aspect, the mutant variants are changes in nucleic acid sequence that change the corresponding 2A amino acid sequence. In one aspect, preferred embodiments of variant 2A sequences are short deletions of the 20 amino acid 2A sequence that retains separating activity. The deletion may comprise removal of about 3 to 6 amino acids at the amino terminus of the 2A region. In another embodiment, Type 2A sequences are mutated by methods well known in the art, such as chemical mutagenensis, oligonucleotide directed mutagenesis, and error prone replication. Mutants with altered separating activity are readily identified by examining expression of the fusion nucleic acids of the present invention. Assaying for production of a separate downstream gene product, such as a reporter protein or a selection protein, allows for identifying sequences having separating activity. Another method for identifying variants may use a FRET based assay using linked GFP molecules, as described above. Insertion of variant 2A sequences in replace of or adjacent to the gly-ser linker region, or other suitable regions linking the GFPs will allow detection of functional 2A separation sequences by identifying constructs that produce separated GFP molecules, as measured by loss of FRET signal. Sequences having no or reduced separating activity will retain higher levels of FRET signal due to physical linkage of the GFP molecules. This strategy will permit high throughput analysis of variants and allows selecting of sequences having high efficiency Type 2A separating activity.

In yet another embodiment, Type 2A separation sequences include homologs present in other nucleic acids, including nucleic acids of other viruses, bacteria, yeast, and multicellular organisms such as worms, insects, birds, and mammals. Homology in this context means sequence similarity or identity. A variety of sequence based alignment methodologies, which are well known to those skilled in the art, are useful in identifying homologous sequences. These include, but not limited to, the local homology algorithm of Smith, F. and Waterman, M. S. (1981) Adv. Appl. Math. 2: 482-89, homology alignment algorithm of Peason, W. R. and Lipman, D. J. (1988) Proc. Natl. Acad. Sci. USA 85: 244448, Basic Local Alignment Search Tool (BLAST) described by Altschul, S. F. et al. (1990) J. Mol. Biol. 215: 403-10, or the Best Fit program described by Devereau, J. et al. (1984) Nucleic Acids. Res. 12: 387-95, and the FastA and TFASTA alignment programs, preferably using default settings or by inspection.

C. Methods of Introducing Nucleic Acids Into Cells

Methods for introducing nucleic acid (e.g., DNA) into cells have been described extensively in the art. Many of these methods can be applied to cells either in vitro or in vivo. Such methods can be used to express, e.g., migration molecules, reporter genes, and/or test agents. Non-limiting examples of techniques which can be used to introduce an expression vector encoding a peptide or antibody of the invention into a host cell include the following.

Naked DNA can be introduced into cells by complexing the DNA to a cation, such as polylysine, which is then coupled to the exterior of an adenovirus virion (e.g., through an antibody bridge, wherein the antibody is specific for the adenovirus molecule and the polylysine is covalently coupled to the antibody) (see Curiel, D. T., et al. (1992) Human Gene Therapy 3:147-154). Entry of the DNA into cells exploits the viral entry function, including natural disruption of endosomes to allow release of the DNA intracellularly. A particularly advantageous feature of this approach is the flexibility in the size and design of heterologous DNA that can be transferred to cells.

Naked DNA can also be introduced into cells by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. Receptors to which a DNA-ligand complex can be targeted include the asialoglycoprotein receptor for hepatocytes, mannose for macrophages (lymphoma), mannose 6-phosphate glycoproteins for fibroblasts (fibrosarcoma), intrinsic factor-vitamin B12 and bile acids (See Kramer et al. (1992) J. Biol. Chem. 267:18598-18604) for enterocytes, insulin for fat cells, and transferrin for smooth muscle cells or other cells bearing transferrin receptors. Additionally, a DNA-ligand complex can be linked to adenovirus capsids which naturally disrupt endosomes, thereby promoting release of the DNA material into the cytoplasm and avoiding degradation of the complex by intracellular lysosomes (see for example Curiel et al. (1991) Proc. Natl. Acad. Sci. USA 88:8850; and Cotten, M. et al. (1992) Proc. Natl. Acad. Sci. USA 89:6094-6098; Wagner, E. et al. (1992) Proc. Natl. Acad. Sci. USA 89:6099-6103). Receptor-mediated DNA uptake can be used to introduce DNA into cells either in vitro or in vivo and, additionally, has the added feature that DNA can be selectively targeted to a particular cell type by use of a ligand which binds to a receptor selectively expressed on a target cell of interest.

Naked DNA can be introduced into cells by mixing the DNA with a liposome suspension containing cationic lipids. The DNA/liposome complex is then incubated with cells. Liposome mediated transfection can be used to stably (or transiently) transfect cells in culture in vitro. Protocols can be found in Current Protocols in Molecular Biology, Ausubel, F. M. et al. (eds.) Greene Publishing Associates, (1989), Section 9.4 and other standard laboratory manuals. Additionally, gene delivery in vivo has been accomplished using liposomes. See for example Nicolau et al. (1987) Meth. Enz. 149:157-176; Wang and Huang (1987) Proc. Natl. Acad. Sci. USA 84:7851-7855; Brigham et al. (1989) Am. J. Med. Sci. 298:278; and Gould-Fogerite et al. (1989) Gene 84:429438. Naked DNA can also be introduced into cells by packaging the DNA into retroviral particles.

Naked DNA can be introduced into cells by directly injecting the DNA into the cells. For an in vitro culture of cells, DNA can be introduced by microinjection, although this not practical for large numbers of cells. Direct injection has also been used to introduce naked DNA into cells in vivo (see e.g., Acsadi et al. (1991) Nature 332: 815-818; Wolff et al. (1990) Science 247:1465-1468). A delivery apparatus (e.g., a “gene gun”) for injecting DNA into cells in vivo can be used. Such an apparatus is commercially available (e.g., from BioRad).

The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to many other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that can integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

Another aspect of the invention pertains to vectors, for example expression vectors, containing a nucleic acid encoding a migration molecule or vectors containing a nucleic acid molecule which encodes a migration polypeptide (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of polypeptide desired, and the like. The expression vectors of the invention can be introduced into host cells to thereby produce, e.g., stably overexpress, proteins or peptides, including fusion proteins or peptides, encoded by nucleic acids as described herein.

Accordingly, an exemplary embodiment provides a method for producing a polypeptide, preferably a migration polypeptide, by culturing in a suitable medium a host cell of the invention (e.g., a mammalian host cell such as a non-human mammalian cell) containing a recombinant expression vector, such that the polypeptide is produced. In a preferred embodiment, the cell stably overexpresses the polypeptide.

The recombinant expression vectors of the invention can be designed for expression of polypeptides in prokaryotic or eukaryotic cells. For example, polypeptides can be expressed in bacterial cells such as E. coli, insect cells (using baculovirus expression vectors) yeast cells or mammalian cells, e.g., such as lymphocytes, e.g., T-cells and B cells or endothelial cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada, et al., (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari, et al., (1987) Embo J 6:229-234), pMFa (Kurjan and Herskowitz, (1982) Cell 30:933-943), pJRY88 (Schultz, et al., (1987) Gene 54:113-123), pYES2 (Invitrogen Corporation, San Diego, Calif.), and picZ (InVitrogen Corp, San Diego, Calif.).

Alternatively, polypeptides can be expressed in insect cells using baculovirus expression vectors. Baculovirus vectors available for expression of proteins in cultured insect cells (e.g., Sf9 cells) include the pAc series (Smith, et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman, et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji, et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci., USA 86:5473-5477), pancreas-specific promoters (Edlund, et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to a migration molecule mRNA. In one embodiment, the migration molecule may be in antisense orientation but translated in the proper orientation from the promoter. For example, the TIM EDG1 vector (set forth in FIG. 1) contains the EDG1 gene in antisense orientation. The protein is translated in the proper orientation from the TRE/pmin promoter.

Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

In one embodiment, of the invention, the cell is transformed with a fusion nucleic acid. In another preferred embodiment, at least one of the genes within the fusion nucleic acid comprises a candidate agent. The candidate agents may be cDNA, fragment of cDNA, genomic DNA fragment, or candidate nucleic acids encoding random or biased random peptides. Expression of fusion nucleic acids where the first gene of interest is a candidate agent and a second gene of interest is a reporter gene allows selection of cells expressing the candidate agent. Alternatively, if the second gene of interest encodes a protein producing a dominant effect, expression of a variety of candidate agents—as a first gene of interest—will permit screening of candidate agents acting as effectors or regulators of the dominantly active protein. By “effector” herein is meant inhibition, activation, or modulation of the cellular phenotype produced by the dominant effect protein. For example, the dominantly acting protein may have a tyrosine kinase activity which activates or inhibits signaling cascades to produce a detectable cellular phenotype. Expression of candidate agents can identify candidate agents acting as kinase inhibitors that suppress the phenotype generated by the protein encoded by the second gene of interest.

As the present invention allows for various combinations of genes of interest within the fusion nucleic acid, one preferred combination is a first and second gene of interest encoding two different reporter/selection proteins. These constructs provide two different basis for detecting a cell expressing the fusion nucleic acid. For example, the first gene of interest may be a GFP and the second gene of interest a β-galactosidase, which permits increased discrimination of cells expressing the fusion nucleic acid by detecting both GFP and β-galactosidase activities. Alternatively, another combination comprises a first gene of interest comprising a migration molecule and a second gene of interest comprising a selection gene. This allows selection for cells expressing fusion nucleic acid based on expression of the selection gene, such as a drug resistance gene (e.g., puromycin), as well as expression of the reporter construct.

When expressing a plurality of genes of interest, there is no particular order of the genes of interest on the fusion nucleic acid. One embodiment may have a first gene of interest upstream of a second gene of interest. Another embodiment may have the second gene of interest upstream and the first gene of interest downstream. By “upstream” and “downstream” herein is meant the proximity to the point of transcription initiation, which is generally localized 5′ to the coding sequence of the fusion nucleic acid. Thus, in a preferred embodiment, the upstream gene of interest is more proximal to the transcription initiation site than the downstream gene of interest.

Methods for generating stably transformed cell lines using retroviral vectors, e.g., self-inactivating (SIN) retroviral vectors, are described in U.S. Patent Publication No. 20040002056, the contents of which are incorporated herein by reference.

Defective retroviruses are well characterized for use in gene transfer for gene therapy purposes (for a review see Miller, A. D. (1990) Blood 76:271). A recombinant retrovirus can be constructed having a nucleic acid encoding a gene of interest (e.g., a gene encoding a peptide or antibody of interest) inserted into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include .psi.Crip, .psi.Cre, .psi.2 and .psi.Am. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Various retroviral vectors are known, including vectors based on the murine stem cell virus (MSCV) (see Hawley, R. G. et al. (1994) Gene Ther. 1: 136-38), modified MFG virus (Riviere, I. et al. (1995) Genetics 92: 6733-37), pBABE (see PCT US97/01019), and pCRU5 (Naviaus, R. K. et al. (1996) J. Virol. 70: 5701-05); all references are hereby expressly incorporated by reference. In addition, particularly well suited retroviral transfection systems for generating retroviral vectors are described in Mann et al., supra; Pear, W. S. et al. (1993) Pro. Natl. Acad. Sci. USA 90: 8392-96; Kitamura, T. et al. (1995) Proc. Natl. Acad. Sci. USA 92: 9146-50; Kinsella, T. M. et al. (1996) Hum. Gene Ther. 7: 1405-13; Hofmann, A. et al. (1996) Proc. Natl. Acad. Sci. USA 93: 5185-90; Choate, K. A. et al. (1996) Hum. Gene Ther. 7: 2247-53; WO 94/19478; PCT U.S. 97/01019, and references cited therein, all of which are incorporated by reference.

In a preferred embodiment, the retroviral vectors are self-inactivating retroviral vectors or SIN vectors. By “self-inactivating” or “SIN” or grammatical equivalents herein is meant retroviral vectors in which the viral promoter elements are rendered ineffective or inactive (see Yu, S.-F. et al. (1986) Proc. Natl. Acad. Sci. USA 83: 3094-84). These promoter and enhancer elements are present in the 3′ long terminal repeat (3′ LTR), which is composed of segments designated as U3 and R (see John M. Coffin, Retroviridae: The Viruses and Their Replication, in Virology, Vol. 2, 1767-1847 (Bernard M. Fields et al. eds.) (3rd ed. 1996). The integrated retroviral genome, called the provirus, is bounded by two LTRs and is transcribed from the 5′ LTR to the 3′ LTR. The viral promoters and enhancers reside generally in the U3 region of the 3′ LTR, but the 3′ LTR region is duplicated at the 5′ LTR during viral integration. Promoter elements situated at the 5′ LTR direct expression of virally encoded genes and generate the RNA copies that are packaged into viral particles.

The self-inactivating feature of SIN vectors arises from the mechanism of viral replication and integration (see Coffin, supra). Following entry of the retrovirus into a cell, a tRNA molecule binds to the primer binding region (PB) at the 5′ end of the viral RNA. Extension of the tRNA primer by reverse transcriptase results in a tRNA linked to a DNA segment containing the U5 and R sequences present at the 5′ end of the viral RNA. RNase activity of reverse transcriptase acts on the viral RNA strand of the DNAIRNA hybrid, thus releasing the elongated tRNA, which then hybridizes to complementary R sequences present on the 3′ end of the viral genome. Elongation by reverse transcriptase results in synthesis of a DNA copy of the viral genome (minus strand DNA) and degradation of the RNA strand by RNase. A short RNA sequence designated the PP sequence, which is resistant to RNase action, remains hybridized to the newly synthesized DNA strand—generally at a region immediately preceding the U3 region at the 3′ end of the viral genome—and acts as a primer for replication of the complementary strand (plus strand DNA). Extension of this PP primer results in replication of sequences comprising U3, R, U5, and PB segments, which eventually become the 5′ LTR of the integrated virus. Subsequently, the PB region of the extended primer hybridizes to the complementary PB region present on the 3′ end of the minus strand DNA, and subsequent extension of this hybrid results in synthesis of a double strand DNA intermediate in which the 5′ and 3′ LTR contain the U3, R, and U5 segments. Following replication and transport into the nucleus, the viral double stranded DNA integrates into the host chromosome via the attachment sites (att) present near the ends of the LTRs, to generate the integrated provirus.

Since the mechanism of viral replication results in duplication of the promoter elements at the 3′ LTR to the 5′ LTR of the integrated virus, inactivating or replacing the viral promoter results in inactivating or replacing the promoter normally present in the proviral 5′ LTR. This feature describes the self-inactivating nature of these retroviral vectors. Inactivation of the 5′ LTR promoter reduces expression of the proviral nucleic acid from the 5′ LTR and reduces the potential deleterious effects arising from influences on cellular genes by the viral promoter present on the 3′ LTR of the integrated virus.

Accordingly, the SIN vectors used in the present invention comprise fusion nucleic acids in which the viral promoter elements, as generally defined below, are rendered inactive or ineffective. By “ineffective” is meant a promoter whose transcriptional activity is reduced by about 80% as compared to promoter activity of the intact viral promoter/enhancer or other measurable promoter activities in the cell. Preferred are reductions in promoter activities of about 90%, with most preferred being inactivation of the viral promoter/enhancer as compared to a cellular promoter or intact viral promoter. By “inactivation” or grammatical equivalents herein is meant that transcription directed by viral sequences in not detected by the assays described below or is about 1% or lower than that of an identifiable promoter activity, such as a constitutively active promoter.

In the present invention, the transformed cells may comprise a plurality of SIN vectors. In one aspect, the plurality of SIN vectors in a cell express different genes of interest. Thus, in one preferred embodiment, at least one SIN vector expresses a candidate agent while at least one other SIN vector expresses gene(s) of interest used for detecting an altered phenotype, e.g., a migration molecule. Alternatively, at least one of the SIN vector expresses a gene of interest which regulates the promoter of another SIN vector in the cell, thus allowing regulated expression of other SIN vectors. In this way, expression of candidate agents may be regulated during the screening process.

Altering the viral promoter/enhancer to render it ineffective or inactive to produce SIN vectors is accomplished by various methods well known to those skilled in the art, e.g., as taught in U.S. Application No. 20040002056 and the references cited therein. These references are incorporated herein by this reference. When an SIN vector expresses separate protein products encoded by the genes of interest, the fusion nucleic acids further comprises separation sequences. By a “separation sequence” or “separation site” or grammatical equivalents as used herein is meant a sequence that results in protein products not linked by a peptide bond. Separation may occur at the RNA or protein level. By being separate does not preclude the possibility that the protein products of the first gene of interest and the second gene of interest interact either non-covalently or covalently following their synthesis. Thus, the separate protein products may interact through hydrophobic domains, protein-interaction domains, common bound ligands, or through formation of disulfide linkages between the proteins.

Various types of separation sequences may be employed. In one embodiment, the separation sequence encodes a recognition site for a protease. A protease recognizing the site cleaves the translated protein product into two or more proteins. Preferred protease cleavage sites and cognate proteases include, but are not limited to, prosequences of retroviral proteases including human immunodeficiency virus protease, and sequences recognized and cleaved by trypsin (EP 578472), Takasuga, A. et al. (1992) J. Biochem. 112: 652-57), proteases encoded by Picornaviruses (Ryan, M. D. et al. (1997) J. Gen. Virol. 78: 699-723), factor Xₐ (Gardella, T. J. et al. (1990) J. Biol. Chem. 265: 15854-59; WO 9006370), collagenase (J03280893; WO 9006370; Tajima, S. et al. (1991) J. Ferment. Bioeng. 72: 362), clostripain (EP 578472), subtilisin (including mutant H64A subtilisin, Forsberg, G. et al. (1991) J. Protein Chem. 10: 517-26), chymosin, yeast KEX2 protease (Bourbonnais, Y. et al. (1988) J. Bio. Chem. 263: 15342-47), thrombin (Forsberg et al., suPra; Abath, F. G. et al. (1991) BioTechniques 10: 178), Staphylococcus aureus V8 protease or similar endoproteinase-Glu-C to cleave after Glu residues (EP 578472; Ishizaki, J. et al. (1992) Appl. Microbiol. Biotechnol. 36: 483-86), cleavage by Nla proteainase of tobacco etch virus (Parks, T. D. et al. (1994) Anal. Biochem. 216: 413-17), endoproteinase-Lys-C (U.S. Pat. No. 4,414,332) and endoproteinase-Asp-N, Neisseria type 2 IgA protease (Pohlner, J. et al. (1992) Biotechnology 10: 799-804), soluble yeast endoproteinase yscF (EP 467839), chymotrypsin (Altman, J. D. et al. (1991) Protein Eng. 4: 593-600), enteropeptidase (WO 9006370), lysostaphin, a polyglycine specific endoproteinase (EP 316748), the family of caspases (e.g., caspase 1, caspase 2, capase 3, etc.), and metalloproteases.

The present invention also contemplates protease recognition sites identified from a genomic DNA, cDNA, or random nucleic acid libraries (see for example, O'Boyle, D. R. et al. (1997) Virology 236: 338-47). For example, the fusion nucleic acids of the present invention may comprise a separation site which is a randomizing region for the display of candidate protease recognition sites. The first and second gene of interest encode reporters molecules useful for detecting protease activity, such as GFP molecules capable of undergoing FRET via linkage through a candidate recognition site (see Mitra, R. D. et al. (1996) Gene;173: 13-7). Proteases are expressed or introduced into cells expressing these fusion nucleic acids. Random peptide sequences acting as substrates for the particular protease result in separate GFP proteins, which is manifested as loss of FRET signal. By identifying classes of recognition sites, optimal or novel protease recognition sequences may be determined.

In addition to their use in producing separate proteins of interest, the protease cleavage sites and the cognate proteases are also useful in screening for candidate agents that enhance or inhibit protease activity. Since many proteases are crucial to pathogenesis of organisms or cellular regulation, for example the HIV or caspase proteases, the ability to express reporter or selection proteins linked by a protease cleavage site allows screens for therapeutic agents directed against a particular protease acting on the recognition site.

Another embodiment of separation sequences are internal ribosome entry sites (IRES), as described herein.

Another aspect of the invention pertains to host cells into which a migration molecule is introduced, e.g., a migration molecule within a vector (e.g., a recombinant expression vector) or a migration nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a polypeptide can be expressed in bacterial cells such as E. coli, insect cells, yeast or mammalian cells (such as lymphocytes, e.g., T-cells and B cells or endothelial cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

Stable cell lines expressing a gene of interest provide significant advantages in studying biological processes and in screens for biologically and pharmacologically active agents. Once isolated, a transformed cell line provides a stable source of gene of interest. There is low variability in expression between cells and all cells express the gene. Uniformly and consistent expression permits facile identification of a cell phenotype when the cells are subjected to a variety of manipulations, for example when exposed to ligands of cell surface receptors. In addition, expressing a gene of interest allows for manipulating the phenotype of cells, which are then useful in identifying agents that alter or change the induced cellular phenotype. These properties afforded by stably transformed cell lines enable large scale screens for candidate agents having biological and pharmacological activity.

Stable cell lines expressing a fusion nucleic acid may be obtained by transient transfection of cells with an expression vector expressing a selectable marker, such as a drug resistance gene. Stable expression relies on non-homologous integration into the chromosome, which is generally random in nature. Optimization of the transfection process for each cell type being analyzed may be required, due to inherent differences in DNA uptake efficiencies.

Stable cell lines expressing genes of interest can also be generated based on homologous recombination mechanisms. Generally described as a “knock-in” or “knock-out” process, the DNA used for recombination have DNA sequences substantially similar to the target sequences on the host chromosome. Recombination between the substantially similar sequences by strand invasions leads to insertion of the nucleic acid vector into the host chromosome.

Stable integration of nucleic acids may also rely on site-specific recombination mediated by recombinases. In these processes, specific recombinases catalyze a reciprocal double-stranded DNA exchange between two DNA segments by recognizing specific sequences present on both partners of the exchange. Specific recombinases are found in both prokaryotes and eukaryotes. In prokaryotes, the .lambda.-integrase acts to insert λ phage into bacterial chromosomes. Similarly transposon integrases, such a γδ resolvase, function to allow integration of transposons into specific sequences within the bacterial genome. Promiscuity of the integration depends on the sequence elements recognized by the resolvase or integrase. Both the resolvase and integrase constitute members of the “tyrosine recombinases” which include flp recombinase of yeast and cre-lox recombinase of P1 bacteriophage.

An analogous system for site specific recombination in eukaryotic cells are the integrases involved in integration of retroviruses. Specificity of integration derives from recognition of specific sequences located at the ends of the linear viral DNA intermediates. The integration is essentially random since insertions occur with high promiscuity, although biases (i.e., hot spots) for particular chromosomal sites are known. After integration, the provirus stably resides in the host chromosome. Consequently, by engineering retroviruses to accommodate non-viral nucleic acids, retroviruses serve as efficient vectors for gene transfer and for creation of cell lines stably transformed with exogenous nucleic acids.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acids encoding a selectable marker can be introduced into a host cell on the same vector as that encoding a migration molecule polypeptide or can be introduced on a separate vector. Cells stably transfected with the introduced nucleic acid can be identified by drug selection (e.g., cells that have incorporated the selectable marker gene will survive, while the other cells die).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of the introduced DNA can be detected, for example, by Northern blotting, RNase protection or reverse transcriptase-polymerase chain reaction (RT-PCR). Expression of the introduced gene product (e.g., the peptide of interest) in the cell can be detected by an appropriate assay for detecting proteins, for example by immunohistochemistry.

As will be appreciated by those skilled in the art, the choice of expression vector system will depend, at least in part, on the host cell targeted for introduction of the nucleic acid. For example, nucleic acids encoding peptides or antibodies of the invention can preferably be administered such that they are expressed in neoplastic cells, e.g., carcinoma cells derived from tissues or organs including breast, testis, ovary, lung, gastrointestinal tract, which spread from one location to another. Alternatively, nucleic acids encoding peptides or antibodies of the invention can be targeted for introduction into cells, such as extracellular matrix cells (connective tissue cells) involved in wound healing, to thereby promote recovery from wounds.

D. Host Cells Expressing Migration Molecules

In one embodiment, the cells used in the instant assays overexpress one or more migration molecules, as described herein. The term “overexpression” as used herein, refers to the expression of a polypeptide, e.g., a migration molecule as described herein, by a cell, at a level which is greater than the normal level of expression of the polypeptide in a cell which normally expresses the polypeptide. For example, expression of the polypeptide may by 5%, 10%, 20%, 30%, 40%, 50%, 60%, 70, 80%, 90%, 100%, or more as compared to expression of the poypeptide in a wild-type cell which normally expresses the polypeptide. In a preferred embodiment, the cells used in the methods of the invention stably overexpress one or more migration molecules.

The cells used in the instant assays can be eukaryotic or prokaryotic in origin. For example, in one embodiment, the cell is a bacterial cell. In another embodiment, the cell is a fungal cell, e.g., a yeast cell. In another embodiment, the cell is a vertebrate cell, e.g., an avian or a mammalian cell. In a preferred embodiment, the cell is a human cell, e.g., immune cells, e.g., T cells and B cells, endothelial cells, fibroblasts, tumor cells, or osteoblasts/osteoclasts. Suitable cells also include known research cells, including, but not limited to, Jurkat T cells, NIH 3T3 cells, CHO, Cos, HeLa, NIH 3T3 etc.

A host cell of the invention, such as a prokaryotic or eukaryotic host cell in culture, can be used to produce (i.e., express) a migration polypeptide. Accordingly, the invention further provides methods for producing a migration polypeptide using the host cells of the invention. In one embodiment, the method comprises culturing the host cell of the invention (into which a recombinant expression vector encoding a migration polypeptide has been introduced) in a suitable medium such that a migration polypeptide is produced. In another embodiment, the method further comprises isolating a migration polypeptide from the medium or the host cell.

E. Test Agents

A variety of test agents can be evaluated using the screening assays described herein. In certain embodiments, the compounds to be tested can be derived from libraries (i.e., are members of a library of compounds). While the use of libraries of peptides is well established in the art, new techniques have been developed which have allowed the production of mixtures of other compounds, such as benzodiazepines (Bunin, et al. (1992). J. Am. Chem. Soc. 114:10987; DeWitt et al. (1993). Proc. Natl. Acad. Sci., USA 90:6909) peptoids (Zuckermann. (1994). J. Med. Chem. 37:2678) oligocarbamates (Cho, et al. (1993). Science. 261:1303), and hydantoins (DeWitt, et al. supra). An approach for the synthesis of molecular libraries of small organic molecules with a diversity of 104-105 as been described (Carell, et al. (1994). Angew. Chem. Int. Ed. Engl. 33:2059; Carell, et al. (1994) Angew. Chem. Int. Ed. Engl. 33:2061).

The compounds of the present invention can be obtained using any of the numerous approaches in combinatorial library methods known in the art, including: biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). Other exemplary methods for the synthesis of molecular libraries can be found in the art, for example in: Erb, et al. (1994). Proc. Natl. Acad. Sci., USA 91:11422-; Horwell, et al. (1996) Immunopharmacology 33:68-; and in Gallop, et al. (1994); J. Med. Chem. 37:1233.

Exemplary compounds which can be screened for activity include, but are not limited to, peptides, nucleic acids, carbohydrates, small organic molecules, and natural product extract libraries.

Candidate/test agents include, for example, 1) peptides such as soluble peptides, including Ig-tailed fusion peptides and members of random peptide libraries (see, e.g., Lam, K. S., et al. (1991) Nature 354:82-84; Houghten, R., et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z., et al. (1993) Cell 72:767-778); 3) antibodies (e.g., antibodies (e.g., intracellular, polyclonal, monoclonal, humanized, anti-idiotypic, chimeric, and single chain antibodies as well as Fab, F(ab′)₂, Fab expression library fragments, and epitope-binding fragments of antibodies); 4) small organic and inorganic molecules (e.g., molecules obtained from combinatorial and natural product libraries); 5) enzymes (e.g., endoribonucleases, hydrolases, nucleases, proteases, synthatases, isomerases, polymerases, kinases, phosphatases, oxido-reductases and ATPases), and 6) mutant forms of molecules.

The test agents of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt, et al. (1993) Proc. Natl. Acad. Sci., U.S.A. 90:6909; Erb, et al. (1994) Proc. Natl. Acad. Sci., USA 91:11422; Zuckermann, et al. (1994) J. Med. Chem. 37:2678; Cho, et al. (1993) Science 261:1303; Carrell, et al. (1994) Angew. Chem. Int. Ed. Engl. 33:2059; Carell, et al. (1994) Angew. Chem. Int. Ed. Engl. 33:2061; and Gallop, et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull, et al. (1992) Proc. Natl. Acad. Sci., USA 89:1865-1869) or phage (Scott and Smith (1990) Science 249:386-390; Devlin (1990) Science 249:404406; Cwirla, et al. (1990) Proc. Natl. Acad. Sci., USA 87:6378-6382; Felici (1991) J. Mol. Biol. 222:301-310; Ladner supra.).

Compounds identified in the subject screening assays may be used, e.g., in methods of modulating cell migration. It will be understood that it may be desirable to formulate such compound(s) as pharmaceutical compositions (described supra) prior to contacting them with cells.

Once a test agent is identified that directly or indirectly modulates cell migration, e.g., modulates the production, expression and/or activity of a gene which regulates cell migration, by one of the variety of methods described herein, the selected test agent can then be further evaluated for its effect on cells, for example by contacting the compound of interest with cells either in vivo (e.g., by administering the compound of interest to a subject) or ex vivo (e.g., by isolating cells from the subject and contacting the isolated cells with the compound of interest or, alternatively, by contacting the compound of interest with a cell line) and determining the effect of the compound of interest on the cells, as compared to an appropriate control (such as untreated cells or cells treated with a control compound, or carrier, that does not modulate the biological response).

Candidate bioactive agents encompass numerous chemical classes, though typically they are organic molecules, preferably small organic compounds having a molecular weight of more than 100 and less than about 2,500 daltons. Candidate agents comprise functional groups necessary for structural interaction with proteins, particularly hydrogen bonding, and typically include at least an amine, carbonly, hydroxyl, or carboxyl group, preferably at least two of them functional chemical groups. The candidate agents often comprise cyclical carbon or heterocyclic structures, and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are proteins, candidate drugs, and other small molecules.

Candidate agents are obtained from a wide variety of sources including libraries of synthetic or natural compounds. For example, numerous means are available for random and directed synthesis of a wide variety of organic compounds and biomolecules, including expression of randomized oligonucleotides (see for example, Gallop, M. A. et al. (1994) J. Med. Chem. 37: 1233-51; Gordon, E. M. et al. (1994) J. Med. Chem. 37:1385-401; Thompson, L. A. et al. (1996) Chem. Rev. 96: 555-600; Balkenhol, F. et al. (1996) Angew. Chem. Int. Ed. 35: 2288-337; and Gordon, E. M. et al. (1996) Acc. Chem. Res. 29: 444-54). Alternatively, libraries of natural compounds in the form of bacterial, fungal, plant and animal extracts are available or readily produced. Additionally, natural or synthetically produced libraries and compounds are readily modified through conventional chemical, physical, and biochemical means. Known pharmacological agents may be subjected to directed or random chemical modifications such as acylation, alkylation, esterification, and amidification to produce structural analogs.

The candidate agent can be pesticides, insecticides or environmental toxins; a chemical (including solvents, polymers, organic molecules, etc); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc); whole cells (including prokaryotic and eukaryotic (including pathogenic cells), including mammalian tumor cells); viruses (including retroviruses, herpes viruses, adenoviruses, lentiviruses, etc.); and spores (e.g., fungal, bacterial, etc.).

One preferred embodiment of candidate agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus, “amino acid” or “peptide residue”, as used herein means both naturally occurring and synthetic amino acids. For example, homo-phenylalanine, citrulline, and norleucine are considered amino acids for the purposes of the invention. “Amino acids” also includes imino residues such as proline and hydroxyproline. The side chains may be either the (R) or (S) configuration. In the preferred embodiment, the amino acids are in the (S) or L configuration. If non-naturally occurring side chains are used, non-amino acid substituents may be used for example to prevent or retard in-vivo degradations. Proteins including non-naturally occurring amino acids may be synthesized or in some cases, made by recombinant techniques (see van Hest, J. C. et al. (1998) FEBS Lett. 428: 68-70 and Tang et al. (1999) Abstr. Pap. Am. Chem. S218: U 138-U 138 Part 2, both of which are expressly incorporated by reference herein).

In a preferred embodiment, the candidate bioactive agents are naturally occurring proteins or fragments of naturally occurring proteins. For example, cellular extracts containing proteins, or random or directed digests of proteinaceous cellular extracts, may be used. In this way, libraries of procaryotic and eukaryotic proteins may be made for screening in the systems described herein. Particularly preferred in this embodiment are libraries of bacterial, fungal, viral, and mammalian proteins, with the latter being preferred, and human proteins being especially preferred.

Candidate agents may encompass a variety of peptidic agents. These include, but are not limited to, (1) immunoglobulins, particularly IgEs, IgGs and IgMs, and particularly therapeutically or diagnostically relevant antibodies, including but not limited to, antibodies to human albumin, apolipoproteins (including apolipoprotein E), human chorionic gonadotropin, cortisol, a-fetoprotein, thyroxin, thyroid stimulating hormone (TSH), antithrombin, antibodies to pharmaceuticals (including antieptileptic drugs (phenyloin, primidone, carbariezepin, ethosuximide, valproic acid, and phenobarbitol), cardioactive drugs (digoxin, lidocaine, procainamide, and disopyramide), bronchodilators (theophylline), antibiotics (chloramphenicol, sulfonamides), antidepressants, immunosuppresants, abused drugs (amphetamine, methamphetamine, cannabinoids, cocaine and opiates) and antibodies to any number of viruses (including orthomyxoviruses, (e.g., influenza virus), paramyxoviruses (e.g., respiratory syncytial virus, mumps virus, measles virus), adenoviruses, rhinoviruses, coronaviruses, reoviruses, togaviruses (e.g., rubella virus), parvoviruses, poxviruses (e.g., variola virus, vaccinia virus), enteroviruses (e.g., poliovirus, coxsackievirus), hepatitis viruses (including A, B and C), herpesviruses (e.g., Herpes simplex virus, varicella-zoster virus, cytomegalovirus, Epstein-Barr virus), rotaviruses, Norwalk viruses, hantavirus, arenavirus, rhabdovirus (e.g., rabies virus), retroviruses (including HIV, HTLV-I and -II), papovaviruses (e.g., papillomavirus), polyomaviruses, and picornaviruses, and the like), and bacteria (including a wide variety of pathogenic and non-pathogenic prokaryotes of interest including Bacillus; Vibrio, e.g., V. cholerae; Escherichia, e.g., Enterotoxigenic E. coli, Shigella, e.g. S. dysenteriae; Salmonella, e.g., S. typhi; Mycobacterium e.g., M. tuberculosis, M. leprae; Clostridium, e.g., C. botulinum, C. tetani, C. difficile, C. perfringens; Cornyebacterium, e.g., C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lamblia Y. pestis, Pseudomonas, e.g. P. aeruginosa, P. putida; Chlamydia, e.g., C. trachomatis; Bordetella, e.g., B. pertussis; Treponema, e.g., T. palladium; and the like); (2) enzymes (and other proteins), including but not limited to, enzymes used as indicators of or treatment for heart disease, including creatine kinase, lactate dehydrogenase, aspartate amino transferase, troponin T, myoglobin, fibrinogen, cholesterol, triglycerides, thrombin, tissue plasminogen activator (tPA); pancreatic disease indicators including amylase, lipase, chymotrypsin and trypsin; liver function enzymes and proteins including cholinesterase, bilirubin, and alkaline phosphatase; aldolase, prostatic acid phosphatase, terminal deoxynucleotidyl transferase, and bacterial and viral enzymes such as HIV protease; (3) hormones and cytokines (many of which serve as ligands for cellular receptors) such as erythropoietin (EPO), thrombopoietin (TPO), the interleukins (including IL-1 through IL-17), insulin, insulin-like growth factors (including IGF-1 and -2), epidermal growth factor (EGF), transforming growth factors (including TGF-α and TGF-β), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cortisol, estradiol, follicle stimulating hormone (FSH), thyroid-stimulating hormone (TSH), luteinizing hormone (LH), progesterone, testosterone; and (4) other proteins (including α-fetoprotein, carcinoembryonic antigen CEA).

In a preferred embodiment, the candidate bioactive agents are peptides of from about 5 to about 30 amino acids, with from about 5 to about 20 amino acids being preferred, and from about 7 to about 15 being particularly preferred. These peptides may be digests of naturally occurring proteins, as described above, or random or biased random peptides and peptide analogs either chemically synthesized or encoded by candidate nucleic acids. By “randomized” or grammatical equivalents herein is meant that each nucleic acid and peptide consists of essentially random nucleotides and amino acids, respectively. Generally, since these random peptides (or nucleic acids, discussed below) are chemically synthesized, they may incorporate any amino acid or nucleotide at any position. The synthetic process can be designed to generate randomized proteins or nucleic acids to allow the formation of all or most of the possible combinations over the length of the sequence, thus forming a library of randomized candidate bioactive proteinaceous agents.

In one embodiment, the library is fully randomized, with no sequence preference or constants at any position. In a preferred embodiment, the library is biased. That is, some positions within the sequence are either held constant or are selected from a limited number of possibilities. For example, in a preferred embodiment, the nucleotides or amino acid residues are randomized within a defined class, for example hydrophobic amino acids, hydrophilic residues, sterically biased (either small or large) residues, or are amino acid residues for crosslinking (e.g., cysteines) or phosphorylation sites (i.e., serines, threonines, tyrosines, or histidines).

In a preferred embodiment, the bias is toward peptides or nucleic acids that interact with known classes of molecules. For example, it is known that much of intracellular signaling is carried out by short regions of polypeptide interacting with other polypeptide regions of other proteins, such as the interaction domains described above. Another example of interaction domain is a short region from the HIV-1 envelope cytoplasmic domain that has been previously shown to block the action of cellular calmodulin. Regions of the Fas cytoplasmic domain, which shows homology to the mastopam toxin from Wasps, can be limited to a short peptide region with death inducing apoptotic or G protein inducing functions. Magainin, a natural peptide derived from Xenopus, can have potent anti-tumor and anti-microbial activity. Short peptide fragments of a protein kinase C isozyme (β-PKC) have been shown to block nuclear translocation of PKC in Xenopus oocytes following stimulation. In addition, short SH-3 target proteins have been used as pseudosubstrates for specific binding to SH-3 proteins. This is of course a short list of available peptides with biological activity, as the literature is dense in this area. Thus, there is much precedent for the potential of small peptides to have activity on intracellular signaling cascades. In addition, agonists and antagonists of any number of molecules may be used as the basis of biased randomization of candidate bioactive agents as well.

Thus, a number of molecules or protein domains are suitable as starting points for generating biased candidate agents. A large number of small molecule domains are known that confer common function, structure or affinity. These include protein-protein interaction domains and nucleic acid interaction domains described above. As is appreciated by those in the art, while variations of these protein-protein or protein-nucleic acid domains may have weak amino acid homology, the variants may have strong structural homology.

In another preferred embodiment, the candidate agents are nucleic acids. By “nucleic acid” or “oligonucleotide” or grammatical equivalents herein is meant at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage, S. L. et al. (1993) Tetrahedron 49: 1925-63 and references therein; Letsinger, R. L. et al. (1970) J. Org. Chem. 35: 3800-03; Sprinzl, M. et al. (1977) Eur. J. Biochem. 81: 579-89; Letsinger, R. L. et al. (1986) Nucleic Acids Res. 14: 3487-99; Sawai et al. (1984) Chem. Left. 805; Letsinger, R. L. et al. (1988) J. Am. Chem. Soc. 110: 4470; and Pauwels et al. (1986) Chemica Scripta 26:141-49), phosphorothioate (Mag, M. et al. (1991) Nucleic Acids Res. 19: 143741; and U.S. Pat. No. 5,644,048), phosphorodithioate (Briu et al. (1989) J. Am. Chem. Soc. 111: 2321), O-methylphophoroamidite linkages (see Eckstein, Oligonucleotides and Analogues: A Practical Approach, Oxford University Press, 1991), and peptide nucleic acid backbones and linkages (Egholm, M. (1992) Am. Chem. Soc. 114:1895-97; Meier et al. (1992) Chem. Int. Ed. Engl. 31:1008; Egholm, M (1993) Nature 365: 566-68; Carlsson, C. et al. (1996) Nature 380: 207, all of which are incorporated by reference). Other analog nucleic acids include those with positive backbones (Dempcy, R. O. et al. (1995) Proc. Natl. Acad. Sci. USA 92: 6097-101); non-ionic backbones (U.S. Pat. Nos. 5,386,023, 5,637,684, 5,602,240, 5,216,141 and 4,469,863; Kiedrowshi et al. (1991) Angew. Chem. Intl. Ed. English 30: 423; Letsinger, R. L. et al. (1988) J. Am. Chem. Soc. 110: 4470; Letsinger, R. L. et al. (1994) Nucleoside & Nucleotide 13: 1597; Chapters 2 and 3, ASC Symposium Series 580, “Carbohydrate Modifications in Antisense Research”, Ed. Y. S. Sanghui and P. Dan Cook; Mesmaeker et al. (1994) Bioorganic & Medicinal Chem. Lett. 4: 395; Jeffs et al. (1994) J. Biomolecular NMR 34: 17; (1996) Tetrahedron Lett. 37: 743) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al. (1995) Chem. Soc. Rev. 169-76). Several nucleic acid analogs are described in Rawls, C & E News Jun. 2, 1997 page 35. All of these references are hereby expressly incorporated by reference. These modifications of the ribose-phosphate backbone may be done to facilitate the addition of additional moieties, such as labels, or to increase the stability and half-life of such molecules in physiological environments. In addition, mixtures of different nucleic acid analogs, and mixtures of naturally occurring nucleic acids and analogs may be made. The nucleic acids may be single stranded or double stranded, as specified, or contain portions of both double stranded or single stranded sequence. The nucleic acid may be DNA, both genomic and cDNA, RNA or hybrid, where the nucleic acid contains any combination of deoxyribo- and ribonucleotides, and any combination of bases, including uracil, adenine, thymine, cytosine, guanine, xanthine hypoxanthine, isocytosine, isoguanine, etc., although generally occurring bases are preferred. In a preferred embodiment, the candidate nucleic acids comprise cDNAs, including cDNA libraries, or fragments of cDNAs. The cDNAs can be derived from any number of different cells and include cDNAs generated from eucaryotic and procaryotic cells, viruses, cells infected with viruses or other pathogens, genetically altered cells, cells with defective cellular processes, etc. Preferred embodiments include cDNAs made from different individuals, such as different patients, particularly human patients. The cDNAs may be complete libraries or partial libraries. Furthermore, the candidate nucleic acids can be derived from a single cDNA source or multiple sources; that is, cDNA from multiple cell types, multiple individuals or multiple pathogens can be combined in a screen. In other aspects, the cDNA may encode specific domains, such as signaling domains, protein interaction domains, membrane binding domains, targeting domains, etc. The cDNAs may utilize entire cDNA constructs or fractionated constructs, including random or targeted fractionation. Suitable fractionation techniques include enzymatic (e.g., DNase I, restriction nucleases etc.), chemical, or mechanical fractionation (e.g., sonicated or sheared). Also useful for the present invention are cDNA libraries enriched for a specific class of proteins, such as type I membrane proteins (Tashiro, K. et al. (1993) Science 261: 600-03) and membrane proteins (Kopczynski, C. C. (1998) Proc. Natl. Acad. Sci. USA 95: 9973-78). Additionally, subtracted cDNA libraries in which genes preferentially or exclusively expressed in particular cells, tissues, or developmental phases are enriched. Methods for making subtracted cDNA libraries are well known in the art (see Diatchenko, L. et al. (1999) Methods Enzymol. 303: 349-80; von Stein, 0. D. et al. (1997) Nucleic Acids Res. 13: 2598-602: Carcinci, P. (2000) Genome Res. 10: 1431-32). Accordingly, a cDNA library may be a complete cDNA library from a cell, a partial library, an enriched library from one or more cell types, or a constructed library with certain cDNAs being removed to from a library. In another preferred embodiment, the candidate nucleic acids comprise libraries of genomic nucleic acids, which includes organellar nucleic acids. As elaborated above for cDNAs, the genomic nucleic acids may be derived from any number of different cells, including genomic nucleic acids of eukaryotes, prokaryotes, or viruses. They may be from normal cells or cells defective in cellular processes, such as tumor suppression, cell cycle control, or cell surface adhesion. Moreover, the genomic nucleic acids may be obtained from cells infected with pathogenic organisms, for example cells infected with viruses or bacteria. The genomic nucleic acids comprise entire genomic nucleic acid constructs or fractionated constructs, including random or targeted fractionation as described above. Generally, for genomic nucleic acids and cDNAs, the candidate nucleic acids may range from nucleic acid lengths capable of encoding proteins of twenty to thousands of amino acid residues, with from about 50-1000 being preferred and from about 100-500 being especially preferred. In addition, candidate agents comprising cDNA or genomic nucleic acids may also be subsequently mutated using known techniques (e.g., exposure to mutagens, error prone PCR, error prone transcription, combinatorial splicing (e.g., cre-lox recombination) to generate novel nucleic acid sequences (or protein sequences). In this way libraries of procaryotic and eukaryotic nucleic acids may be made for screening in the systems described herein. Particularly preferred in the embodiments are libraries of bacterial, fungal, viral and mammalian nucleic acids, with the latter being preferred, and human nucleic acids being especially preferred.

In another preferred embodiment, the candidate nucleic acids comprise libraries of random nucleic acids. Generally, the random nucleic acids are fully randomized or they are biased in their randomization, e.g. in nucleotide/residue frequency generally or per position. As defined above, by “randomized” or grammatical equivalents herein is meant that each nucleic acid consists essentially of random nucleotides. Since the candidate nucleic acids are chemically synthesized, they may incorporate any nucleotide at any position. In the expressed random nucleic acid, at least 10, preferably at least 12, more preferably at least 15, most preferably at least 21 nucleotide positions need to be randomized. The candidate nucleic acids may also comprise nucleic acid analogs as described above.

For candidate nucleic acids encoding peptides, the candidate nucleic acids generally contain cloning sites which are placed to allow in-frame expression of the randomized peptides, and any fusion partners, if present, such as presentation structures.

In a preferred embodiment, the fusion nucleic acids of the present invention further comprises genes of interest linked to a fusion partner to form a fusion polypeptide. By fusion partner or functional group herein is meant a sequence that is associated with the gene of interest, or candidate agent, that confers upon all members of the library in that class a common function or ability. Fusion partners can be heterologous (i.e., not native to the host cell), or synthetic (i.e., not native to any cell). Suitable fusion partners include, but are not limited to: (a) presentation structures, as defined below, which provide the peptides of interest and candidate agents in a conformationally restricted or stable form; (b) targeting sequences which allow the localization of the genes of interest and candidate agent into a subcellular or extracellular compartment; (c) rescue sequences which allow the purification or isolation of either the peptide of interest (for example, when a gene of interest encodes a peptide) or candidate agents or the nucleic acids encoding them; (d) stability sequences, which affects the stability or degradation to the protein of interest or candidate agent or the nucleic acid encoding it, for example resistance or susceptibility to proteolytic degradation; (e) dimerization sequences, to allow for peptide dimerization; or (f) any combination of the above, as well as linker sequences as needed.

In a preferred embodiment, the fusion partner is a presentation structure. By “presentation structure” or grammatical equivalents herein is meant a sequence, when fused to a peptide encoded by gene of interest or peptide candidate agents, causes the peptides to assume a conformationally restricted form. Proteins interact with each other largely through conformationally constrained domains. Although small peptides with freely rotating amino and carboxyl termini can have potent functions as is known in the art, the conversion of such peptide structures into pharmacologic or biologically active agents is difficult due to the inability to predict side-chain positions for peptidomimetic synthesis. Therefore the presentation of peptides in conformationally constrained structures will benefit both the later generation of pharmaceuticals and will also likely lead to higher affinity interactions of the peptide with the target protein. This fact has been recognized in the combinatorial library generation systems using biologically generated short peptides in bacterial phage systems. A number of workers have constructed small domain molecules in which one might present short peptide domains or randomized peptide structures.

Presentation structures are preferably used with peptides encoded by genes of interest and peptide candidate agents encoded by random nucleic acids, although candidate agents, may be either nucleic acid or peptides. Thus, when presentation structures are used with peptide candidate agents, synthetic presentation structures, i.e., artificial polypeptide, are adaptable for presenting a peptide, for example a randomized peptide, as a conformationally-restrict-ed domain. Generally, such presentation structures comprise a first portion joined to the N-terminal end of the peptide, and a second portion joined to the C-terminal end of the peptide; that is, the peptide is inserted into the presentation structure, although variations may be made, as outlined below. To increase the functional isolation of the peptide expression product, the presentation structures are selected or designed to have minimal biologically activity when expressed in the target cell.

Preferred presentation structures maximize accessibility to the peptide by presenting it on an exterior loop. Accordingly, suitable presentation structures include, but are not limited to, minibody structures, loops on β-sheet turns and coiled-coil stem structures in which residues not critical to structure are randomized, zinc-finger domains, cysteine-linked (disulfide) structures, transglutaminase linked structures, cyclic peptides, B-loop structures, helical barrels or bundles, leucine zipper motifs, etc.

Examples of presentation structures, targeting sequences, rescue sequences, stability sequences and dimerization sequences are set forth in U.S. Patent Application 20040002056, the contents of which are incorporated herein by reference.

For example, when presentation structures are used, the presentation structure will generally contain the initiating ATG as part of the parent vector. For candidate agents comprising RNAs, in addition to chemically synthesized RNA nucleic acids, the candidate nucleic acids may be expressed from vectors, including retroviral vectors. Thus, when the RNAs are expressed, vectors expressing the candidate nucleic acids may be constructed with an internal promoter (e.g., CMV promoter), tRNA promoter, cell specific promoter, or hybrid promoters designed for immediate and appropriate expression of the RNA structure at the initiation site of RNA synthesis. For retroviral vectors, the RNA may be expressed anti-sense to the direction of retroviral synthesis and is terminated as known, for example with an orientation specific terminator sequences. Interference from upstream transcription is minimized in the target cell by using the SIN vectors described herein.

When the nucleic acids are expressed in the cells, they may or may not encode a protein as described herein. Thus, included within the candidate nucleic acids of the present invention are RNAs capable of producing an altered phenotype. In this regard, the nucleic acid may be an antisense RNA directed towards a complementary target nucleic acid, RNAs capable of catalyzing cleavage of target nucleic acids in a sequence specific manner, preferably in the form of ribozymes (e.g., hammerhead ribozymes, hairpin ribozymes, and hepatitis delta virus ribozymes), and double stranded RNA capable of inducing RNA interference or RNAi, as described above.

In a preferred embodiment, a library of candidate bioactive agents are used. Preferably, the library should provide a sufficiently structurally diverse population of randomized expression products to effect a probabilistically sufficient range to provide one or more peptide products which has the desired properties such as binding to protein interaction domains or producing a desired cellular response. For example, in the case of libraries of random peptides, a library must be large enough so that at least one of its members will have a structure that gives it affinity for some molecule, protein or other factor whose activity is involved in some cellular response, such as signal transduction. Although it is difficult to gauge the required absolute size of an interaction library, nature provides a hint with the immune response: a diversity of 10⁷-10⁸ different antibodies provides at least one combination with sufficient affinity to interact with most potential antigens faced by an organism.

Published in vitro selection techniques have also shown that a library size of about 10⁶ to 10⁸ is sufficient to find structures with affinity for the target. A library of all combinations of a peptide 7-20 amino acids in length, such as proposed here for expression in retroviruses, has the potential to code for 20⁷ (10⁹) to 20²⁰. Thus with libraries of 10⁷ to 10⁸ per ml of retroviral particles the present methods allow a “working” subset of a theoretically complete interaction library for 7 amino acids, ad a subset of shapes for the 20²⁰ library. Thus in a preferred embodiment, at least 10⁶, preferably at least 10⁷, more preferably at least 10⁸, and most preferably at least 10⁹ different expression products are simultaneously analyzed in the subject methods. Preferred methods maximize library size and diversity.

The candidate bioactive agents are combined, added to, or contacted with a cell or population of cells or plurality of cells. By “population of cells” or “plurality of cells” herein is meant at least two cells, with at least about 10⁵ being preferred, at least about 10⁶ being particularly preferred, and at least about 10⁷, 10⁸, and 10⁹ being especially preferred.

The candidate agents and the cells are combined. As will be appreciated by those in the art, this may be accomplished in any number of ways, including adding the candidate agents to the surface of the cells, to the media containing the cells, or to a surface on which the cells grow or contact. The candidate agents and cells may be combined by adding the agents into the cells, for example by using vectors that will introduce agents into the cells, especially when the candidate agents are nucleic acids or proteins.

In a preferred embodiment, the candidate agents are either nucleic acids or proteins that are introduced into the cells to screen for candidate agents capable of altering the phenotype of a cell. By “introduced into” or grammatical equivalents herein is meant that the nucleic acids enter the cells in a manner suitable for subsequent expression of the nucleic acid. The method of introduction is largely dictated by the targeted cell type, discussed below. Exemplary methods include CaPO.sub.4 transfection, DEAE dextran transfection, liposome fusion, lipofectin.RTM.), electroporation, viral infection, biolistic particle bombardment etc. The candidate nucleic acids may exist either transiently or stably in the cytoplasm or stably integrate into the genome of the host cell (i.e., by retroviral integration). As many pharmaceutically important screens require human or model mammalian cell targets, retroviral vectors capable of transfecting such targets are preferred.

In a preferred embodiment, the candidate bioactive agents are either nucleic acids or proteins (proteins in this context includes proteins, oligopeptides, and peptides) that are expressed in the host cells using vectors, including viral vectors. The choice of the vector, preferably a viral vector, will depend on the cell type. When cells are replicating, retroviral vectors are used. When the cells are not replicating, for example when arrested in one of the growth phases, viral vectors capable of infecting non-dividing cells, including lentiviral and adenoviral vectors, are used to express the nucleic acids and proteins.

In a preferred embodiment, the candidate bioactive agents are either nucleic acids or proteins that are introduced into the host cells using retroviral vectors, as is generally outlined in PCT U.S. 97/01019 and PCT US97/01048, both of which are expressly incorporated by reference. Generally, a library is generated using a retroviral vector backbone. For generating a random nucleic acid or peptide library, standard oligonucleotide synthesis is done to generate the nucleic acids. After synthesizing the nucleic acid library, the library is cloned into a first primer, which serves as a cassette for insertion into the retroviral construct. The first primer generally contains additional elements, including for example, the required regulatory sequences (e.g., translation, transcription, promoters, etc.) fusion partners, restriction endonuclease sites, stop codons, regions of complementarity for second strand priming.

A second primer is then added, which generally consists of some or all of the complementarity region to prime the first primer and optional sequences necessary to a second unique restriction site for purposes of subcloning. Extension with DNA polymerase results in double stranded oligonucleotides, which are then cleaved with appropriate restriction endonucleases and subcloned into the target retroviral vectors.

When the candidate agents are cDNAs or genomic DNAs, these nucleic acids are inserted into the retroviral vector by methods well known in the art. The DNAs may be inserted unidirectionally or randomly using appropriate adaptor sequences and vector restriction sites.

Any number of suitable retroviral vectors may be used. In one aspect, preferred vectors include those based on murine stem cell virus (MSCV) (Hawley, et al. (1994) Gene Therapy 1: 136), a modified MFG virus (Reivere et al. (1995) Genetics 92: 6733), pBABE, and others described above. Well suited retroviral transfection systems are described in Mann et al, supra; Pear et al. (1993) Proc. Natl. Acad. Sci. USA 90: 8392-96; Kitamura, et al. Human Gene Ther. 7: 1405-1413; Hofmann, et al Proc. Natl. Acad. Sci. USA 93: 5185-90; Choate et (1996) Human Gene Ther 7: 2247; WO 94/19478; PCT US97/01019, and references cited therein, all of which are incorporated by reference.

In one preferred embodiment, the retroviral vectors used to introduce candidate agents comprise the SIN vectors described herein.

III. Pharmaceutical Compositions and Administration

The modulators identified by the screening assays of the invention can be incorporated into pharmaceutical compositions suitable for administration. Such compositions typically comprise the nucleic acid molecule, or polypeptide and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Pharmaceutically acceptable carriers are determined in part by the particular composition being administered (e.g., nucleic acid, protein, modulatory compounds or transduced cell), as well as by the particular method used to administer the composition.

A pharmaceutical composition of the invention is formulated to be compatible with its intended route of administration. Examples of routes of administration include parenteral, e.g., intravenous, by intraarticular (in the joints), intramuscular, intradermal, intraperitoneal, subcutaneous, oral (e.g., inhalation), transdermal (topical), transmucosal, vaginal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound or transduced cell in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery. Vaginal suppositories or foams for local mucosal delivery may also be prepared to block sexual transmission.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens and liposomes targeted to macrophages containing, for example, phosphatidylserine) can also be used as pharmaceutically acceptable carriers. These can be prepared according to methods known to those skilled in the art, for example, as described in U.S. Pat. No. 4,522,811 and U.S. Pat. No. 5,643,599, the entire contents of which are incorporated herein.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. Cells transduced by nucleic acids for ex vivo therapy can also be administered intravenously or parenterally as described above.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test agent which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease, disorder, or infection, previous treatments, the general health and/or age of the subject, and other diseases present. Moreover, treatment of a subject with a therapeutically effective amount of a polypeptide or antibody can include a single treatment or, preferably, can include a series of treatments.

In a preferred example, a subject is treated with antibody or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody or polypeptide used for treatment may increase or decrease over the course of a particular treatment. Changes in dosage may result and become apparent from the results of diagnostic assays as described herein.

An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e.,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Amon, et al., “Monoclonal Antibodies For Immunotargeting Of Drugs In Cancer Therapy”, in Monoclonal Antibodies And Cancer Therapy, Reisfeld, et al. (eds.), pp. 243-56 (Alan R. Liss, Inc. 1985); Hellstrom, et al., “Antibodies For Drug Delivery”, in Controlled Drug Delivery (2nd Ed.), Robinson, et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin, et al. (eds.), pp. 303-16 (Academic Press 1985), and Thorpe, et al., “The Preparation And Cytotoxic Properties Of Antibody-Toxin Conjugates”, Immunol. Rev. 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen, et al. (1994) Proc. Natl. Acad. Sci., USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system. In general, the dose equivalent of a naked nucleic acid from a vector is from about 1 μg to 100 μg for a typical 70 kilogram patient, and doses of vectors which include a retroviral particle are calculated to yield an equivalent amount of therapeutic nucleic acid.

### IV. Other Embodiments

In addition to the other embodiments, aspects and objects of the present invention disclosed herein, including the claims appended hereto, the following paragraphs set forth additional, non-limiting embodiments and other aspects of the present invention:

Provided is a method for identifying a compound which modulates cell migration comprising: a) contacting a cell which overexpresses a migration molecule with a test agent and a migration molecule ligand; b) measuring migration of said cell towards said ligand wherein cell migration is modulated in the presence of the test agent as compared to in the absence of the test agent. In certain embodiments, the cells are labeled. and may be optionally labeled with a fluorescent dye, such as CyQuant GR™ dye. The migration may be measured using a fluorescence plate reader. For example, the migration may be measured at 485/530 nm. The compounds may, for example, inhibit cell migration or stimulate cell migration. The method may be carried out in a vessel capable of holding multiple samples, for example, in a 96-well plate. Each well may contain a different test agent.

Further provided is a vector comprising a 5′ long terminal repeat (LTR), a reporter gene, the coding sequence of EDG1, a transcriptional response element (TRE), and a 3′ self-inactivating long terminal repeat (SIN-LTR). The vector may further comprise an internal ribosome entry site (IRES) inserted between the reporter gene and the coding sequence of EDG1. In certain embodiments, the transcriptional response element (TRE) is a minimal promoter (Pmin). In certain embodiments, the reporter gene is GFP.

Still further provided is a vector comprising an EF-1α promoter, a reporter gene, the coding sequence of EDG3, and a marker gene. In certain embodiments, the marker gene is a resistance gene, for example, neomycin. In certain embodiments, the reporter gene is GFP. The vector may further comprise an internal ribosome entry site (IRES) inserted between the reporter gene and the coding sequence of EDG 1.

Such vectors may be used to stably transfect cells, which such cells are also provided herein. Exemplary cells include, but are not limited to, Jurkat cells, lymphocytes and endothelial cells.

### EXAMPLES

This invention is further illustrated by the following examples which should not be construed as limiting. All publications, figures, patents and patent applications mentioned herein are hereby incorporated by reference in their entireties as if each individual publication, figure, patent or patent application was specifically and individually indicated to be incorporated by reference. In case of conflict, the present application, including any definitions herein, will control. Also incorporated by reference in their entireties are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

### EXAMPLE 1

### Assay for Measuring EDG1 and EDG3-Mediated Cell Migration

In order to develop an assay to measure EDG1 and EDG3-mediated cell migration, two T lymphoid cell lines were generated which overexpress EDG1 and EDG3 respectively (see FIG. 1). Both cell lines exhibit enhanced migration activity toward S1P. EDG1 expression is regulated by tetracycline. In the presence of doxycycline, the EDG1-mediated migration is abolished. EDG3 is constituitively expressed.

Both cell lines were used to optimize assay parameters in order to maximize accuracy and throughput in an automated 96-well format. Compounds were added together with S1P in bottom receiver plate; lipid starved cells were placed in the upper filter plate. The T cell migration may be finished in 2 to 4 hours. The cells which migrated to the receiver plate were stained with a fluorescence dye (CyQuant GR™) and detected by a fluorescence plate reader.

FTY720 (EDG1 angonist) and suramin (EDG3 antagonist) were tested in the assay. Both compounds specifically inhibit EDG1 or EDG3-mediated T cell migration in response to S1P stimuli. This assay greatly simplifies the functional analysis of EDGs-mediated migration and provides a high throughput assay for screening and identifying compounds that block or enhance EDG1 and EDG3-mediated T cell migration.

Migration Assay Employing EDG1 as a Migration Molecule

On day one, EDG1 expressing cells were grown at 0.4 millions/ml in RPMI 1640 containing 10% lipid-free serum (Charcoal-Dextran-Stripped Fetal Bovine Serum, Cat# 100-502 (Gemini Bioproducts™)) for 24 hours. EDG1 cells are maintained below 1 million/ml at all times. On the next day, cells were spun down, washed once with serum-free medium and re-suspended in RPMI 1640 containing 0.1% BSA (fat-free, cell culture tested) (Cat# A8806 (SIGMA ALDRICH FLUKA CHEMICALS™)) at a density of 6×10⁶ per ml. 170 μl RPMI 1640 (0.1% BSA) containing 20 nM S1P (Cat# SL-140 (BioMol™)) and/or desired drugs were added to the receiver plate of 3 μM Millipore Multiscreen MIC™ plates (Cat# MAMI C3S 10, 3 um pore size (Millipore™)) and the filter plates were carefully placed over the receiver plate. 50 μl cell suspension was added to the upper wells of the filter plate. The cells were incubated in a tissue culture incubator at 37° C. for 4 hours. The top filter plate was removed and after a brief agitation, 50 μl of media are transferred from the receiver plate to a white plate and 50 μl 2× Lysis Buffer containing CyQuant GR dye (1: 150 dilution) was added (CyQUAN™ cell proliferation assay, Cat# C-7026 (Molecular Probes™)). After 30 min agitating at RT, the plate was read in a fluorescence plate reader using 480/520 nm filter set.

Migration Assay Employing EDG3 as a Migration Molecule

On day one, EDG3 expressing cells were grown at 0.8 millions/ml in RPMI 1640 containing 10% lipid-free serum (Charcoal-Dextran-Stripped Fetal Bovine Serum, Cat# 100-502 (Gemini Bioproducts™)) for 24 hours. EDG3 cells are maintained below 3 million/ml at all times. On the next day, cells were spun down, washed once with serum-free medium and re-suspended in RPMI 1640 containing 0.1% BSA (fat-free, cell culture tested) (Cat# A8806 (SIGMA ALDRICH FLUKA CHEMICALS™)) at a density of 8×10⁶ per ml. 170 μl RPMI 1640 (0.1% BSA) containing 60 nM S1P (Cat# SL-140 (BiOMOl™)) and/or desired drugs were added to the receiver plate of 3 μM Millipore Multiscreen MIC plates (Cat# MAMI C3S 10, 3 um pore size (Millipore™)) and the filter plates were carefully placed over the receiver plate. 50 μl cell suspension was added to the upper wells of the filter plate. The cells were incubated in a tissue culture incubator at 37° C. for 2 hours. The top filter plate was removed and after a brief agitation, 50 μl of media are transferred from the receiver plate to a white plate and 50 μl 2× Lysis Buffer containing CyQuant GR™ dye (1:150 dilution) was added (CyQUANT™ cell proliferation assay, Cat# C-7026 (Molecular Probes™)). After 30 min agitating at RT, the plate was read in a fluorescence plate reader using 480/520 nm filter set.

Equivalents

Those skilled in the art will recognize, or be able to ascertain using no more than routine experimentation, many equivalents to the invention described in this specification and the claims below. While specific embodiments of the subject invention have been discussed, the above specification is illustrative and not restrictive. For example, variants on the quantities of reactants given in the above Examples are within the scope of the invention, as are variants on the incubation time. The full scope of the invention should be determined by reference to the claims, along with their full scope of equivalents, and the specification, along with such variations.

We claim:

1. A method for identifying a compound which modulates cell migration comprising:
a) contacting a cell which overexpresses a migration molecule with a test agent and a migration molecule ligand;
b) measuring migration of said cell towards said ligand
wherein cell migration is modulated in the presence of the test agent as compared to in the absence of the test agent.

2. The method of claim 1, wherein said cell stably overexpresses a migration molecule.

3. The method of claim 1, wherein said cell transiently overexpresses a migration molecule.

4. The method of claim 1, wherein said cell is a lymphocyte.

5. The method of claim 1, wherein said cell is an endothelial cell.

6. The method of claim 1, wherein said cell is a Jurkat cell.

7. The method of claim 1, wherein said migration molecule is EDG1 or EDG3.

8. The method of claim 1, wherein said migration molecule is selected from the group consisting of: a selectin molecule, an integrin molecule, a cadherin molecule, an immunoglobulin superfamily molecule or a chemokine receptor molecule.

9. The method of claim 8, wherein said chemokine receptor molecule is selected from the group consisting of: CCR1, CCR2, CCR3, CCR4, CCR5, CCR6, CCR7, CCR8, CCR9, CCR10, CCR11, CXCR1, CXCR2, CXCR3, CXCR4, CXCR5, CX3CR1, and XCR1.

10. The method of claim 8, wherein said selectin molecule is selected from the group consisting of: L-selectin, E-selectin, and P-selectin.

11. The method of claim 8, wherein said integrin molecule is selected from the group consisting of: α1β1, α2β1, α3β1, α4β1, α5β1, α6β1, α7β1, α8β1 (VLA-8), α9β1, αvβ3, αVβ1, αLβ2, αMβ2, αXβ2, αIIβ3, α6β3, α6β4, αVβ6, αVβ6, αVβ8, α4β7, αIELβ7, and α11.

12. The method of claim 8, wherein said cadherin molecule is selected from the group consisting of: Cadherin E (1), Cadherin N (2), Cadherin BR (12), Cadherin P (3), Cadherin R (4), Cadherin M (15), Cadherin VE (5) (CD144), Cadherin T & H (13), Cadherin OB (11), Cadherin K (6), Cadherin 7, Cadherin 8, Cadherin KSP (16), Cadherin LI (17), Cadherin 18, Cadherin, Fibroblast 1 (19), Cadherin Fibroblast 2 (20), Cadherin Fibroblast 3 (21), Cadherin 23, Desmocollin 1, Desmocollin 2, Desmoglein 1, Desmoglein 2, Desmoglein 3, and Protocadherin 1, 2, 3, 7, 8, and 9.

13. The method of claim 8, wherein said immunoglobulin superfamily molecule is selected from the group consisting of: Inter-Cellular Adhesion Molecule-1 (I-CAM-1) (CD54), Inter-Cellular Adhesion Molecule-2 (I-CAM-2) (CD102), Inter-Cellular Adhesion Molecule-3 (I-CAM-3) (CD50), and Vascular-Cell Adhesion Molecule (V-CAM), ALCAM (CD166), Basigin (CD147), BL-CAM (CD22), CD44, Lymphocyte function antigen-2 (LFA-2) (CD2), LFA-3 (CD 58), Major histocompatibility complex (MHC) molecules, MAdCAM-1, and PECAM (CD31).

14. The method of claim 8, wherein said ligand is sphingosine-1-phosphate (S1P).

15. The method of claims 1, wherein said cell is lipid starved.

16. The method of claim 1, wherein said cell contains a retroviral vector encoding said migration molecule.

17. The method of claim 7, wherein said cell contains a vector comprising a 5′ long terminal repeat (LTR), a reporter gene, the coding sequence of EDG1, a transcriptional response element (TRE), and a 3′ self-inactivating long terminal repeat (SIN-LTR).

18. The method of claim 17, wherein an internal ribosome entry site (IRES) is inserted between the reporter gene and the coding sequence of EDG1.

19. The method of claim 17, wherein said transcriptional response element (TRE) is a minimal promoter (Pmin).

20. The method of claim 17, wherein said reporter gene is GFP.

21. The method of claim 6, wherein said cell contains a vector comprising an EF-1α promoter, a reporter gene, the coding sequence of EDG3, and a marker gene.

22. The method of claim 21, wherein said marker gene is a resistance gene.

23. The method of claim 22, wherein said resistance gene encodes for neomycin resistance.

24. The method of claim 21, wherein said reporter gene is GFP.

25. The method of claim 21, wherein an internal ribosome entry site (IRES) is inserted between the reporter gene and the coding sequence of EDG1.

26. The method of claim 1, wherein said test agent is selected from the group consisting of: a small organic molecule, polypeptide, antibody, nucleic acid, or lipid.

27. The method of claim 1, wherein said cells are labeled.

28. The method of claim 27, wherein herein said cells are labeled with a fluorescent dye.

29. The method of claim 1, wherein said migration is measured using a fluorescence plate reader.

30. The method of claim 1, wherein the cells are labeled after migration.

31. The method of claim 1, wherein the cells are labeled prior to migration.

32. The method of claim 1, wherein said method is carried out in a high-throughput format.

33. The method of claim 32, wherein said high throughput format is automated.

34. The method of claim 1, wherein said method is carried out in a vessel capable of holding multiple samples.

35. The method of claim 1, wherein said migration is from a first vessel to a second vessel.

36. The method of claim 35, wherein said migration is across a membrane.


SEQUENCE LISTING (3 sequences)

SEQ ID NO: 1 - protein, 22 aa, Artificial Sequence
  Description of Artificial Sequence Synthetic peptide
XXXXXXXXXXLXXXDXEXNPGP

SEQ ID NO: 2 - protein, 5 aa, Artificial Sequence
  Description of Artificial Sequence Synthetic cell-surface receptor binding motif
GRGDS

SEQ ID NO: 3 - protein, 6 aa, Artificial Sequence
  Description of Artificial Sequence Synthetic peptide linker
GGGGGG